(19) United States
(12) Patent Application Publication
(10) Pub. No.: US 2025/0182901 A1
(43) Pub. Date: Jun. 5, 2025

(54) SYSTEMS AND METHODS FOR AFFECTING WELL-BEING

(71) Applicant: Diamond Therapeutics Inc., Toronto (CA)

(72) Inventors: William James TYLER, Chelsea, AL (US); Judith BLUMSTOCK, Toronto (CA); Rhea MEHTA, Mississauga (CA); Joel LIEDERMAN, Thornhill (CA)

(21) Appl. No.: 18/840,679

(22) PCT Filed: Sep. 7, 2022
(86) PCT No.: PCT/IB2022/000513

§ 371 Date: Aug. 22, 2024

Related U.S. Application Data
(60) Provisional application No. 63/317,473, filed on Mar. 7, 2022.

(51) Int. Cl. G16H 50/30; G16H 10/20; G16H 20/10; G16H 20/70; G16H 50/70

(52) U.S. Cl. CPC G16H 50/30; G16H 10/20; G16H 20/10; G16H 20/70; G16H 50/70

(57) ABSTRACT

Disclosed herein are systems and methods for affecting well-being. The systems and methods may comprise receiving data, processing data, and initializing one or more models to identify a state of a user's well-being.

### CROSS-REFERENCE

This application claims benefit of U.S. Provisional Patent Application No. 63/317,473 filed on Mar. 7, 2022, which is incorporated herein by reference in its entirety.

### BACKGROUND

Managing and successfully treating physical, mental, emotional, neurological, cognitive, and social disorders is becoming increasingly more difficult as more and more people are suffering from such disorders. For example, published data shows that during the coronavirus of 2019 (COVID-19) pandemic, rates of depression and anxiety were about 4 times as high in adults as before the pandemic. In addition, an increase in instances of physical illness poses an additional risk to the health and well-being of those suffering. Further, published data shows there are several challenges associated with measuring improvements and the quality of people's well-being who suffer from such disorders. There therefore exists an increased demand for effectively affecting and improving the well-being of those suffering with physical, mental, emotional, neurological, cognitive, and social disorders.

### SUMMARY

The present disclosure provides systems and methods for affecting a user's well-being. Provided in certain embodiments herein is a method for affecting a user's well-being. In some embodiments, the method comprises receiving data from one or more data sources. In some embodiments, the method further comprises processing the data received from the one or more data sources to generate or extract one or more user specific parameters from the received data. In some embodiments, the method further comprises initializing one or more models of well-being incorporating data from at least a reference population or a historical user database. In some embodiments, the method further comprises generating an output indicative of a state of the user's well-being based at least in part on the user specific parameters and the data from at least the reference population or the historical user database using the one or more models. In some embodiment, the method comprises: (a) receiving data from one or more data sources; (b) processing the data received from the one or more data sources to generate or extract one or more user specific parameters from the received data; and (c) initializing one or more models of well-being incorporating data from at least a reference population or a historical user database, and generating an output indicative of a state of the user's well-being based at least in part on the user specific parameters and the data from at least the reference population or the historical user database using the one or more models.

Provided in some embodiments herein is a method for affecting a user's well-being comprising: (a) initializing one or more models of well-being incorporating data from at least a reference population or a historical user database, (b) generating user specific parameters relating to the user's well-being, and (c) simulating the user's well-being based at least in part on the user specific parameters and the reference population data or the data in the historical user database. In some embodiments, the method may further comprise (d) updating the one or more models based on newly received data from the user in (b) and newly received data from the reference population or the historical user database. In some embodiments, the method may further comprise generating one or more recommendations to the user to affect (e.g., improve) the user's well-being.

Provided in some embodiments herein is a computer-implemented method for affecting a user's well-being, comprising (a) receiving data from one or more data sources; (b) processing the data received from the one or more data sources to generate or extract one or more user specific parameters from the received data; and (c) initializing one or more models of well-being incorporating data from at least a reference population or a historical user database, and generating an output indicative of a state of the user's well-being based at least in part on the user specific parameters and the data from at least the reference population or the historical user database using the one or more models.

Provided in some embodiments herein are one or more non-transitory computer storage media storing instructions that are operable, when executed by one or more computers, to cause the one or more computers to perform operations comprising: (a) receiving data from one or more data sources; (b) processing the data received from the one or more data sources to generate or extract one or more user specific parameters from the received data; and (c) initializing one or more models of well-being incorporating data from at least a reference population or a historical user database, and generating an output indicative of a state of the user's well-being based at least in part on the user specific parameters and the data from at least the reference population or the historical user database using the one or more models.

Provided in some embodiments herein is a system comprising one or more computers and one or more storage devices storing instructions that are operable, when executed by the one or more computers, to cause the one or more computers to perform operations comprising: (a) receiving data from one or more data sources; (b) processing the data received from the one or more data sources to generate or extract one or more user specific parameters from the received data; and (c) initializing one or more models of well-being incorporating data from at least a reference population or a historical user database, and generating an output indicative of a state of the user's well-being based at least in part on the user specific parameters and the data from at least the reference population or the historical user database using the one or more models.

In any of the embodiments provided herein, affecting the user's well-being comprises improving the user's well-being. In any of the embodiments provided herein, affecting the user's well-being comprises improving the user's health outcomes. In any of the embodiments provided herein, improving the user's health outcomes comprises improving a user's state in regards to one or more of the following emotions: alertness, excitement, elation, happiness, contentment, relaxation, calmness, sleepiness, fatigue, boredom, depression, sadness, frustration, stress, nervousness, tenseness, or a combination thereof. In any of the embodiments provided herein, improving the user's health outcomes can comprise improving a user's ability to cope with or overcome a disorder or condition, for example, a neurological condition the user is suffering from. In any of the embodiments provided herein, the neurological condition is a neurological disorder. In some embodiments, the neurological condition is a neurocognitive disorder. In some embodiments, the symptoms of the neurological condition are physical, behavioral, emotional, mental or a combination thereof. In some embodiments, the neurological condition is an addictive disorder. In some embodiments, the addictive disorder is alcohol abuse, substance abuse, smoking, or obesity. In some embodiments, the neurological condition is an eating disorder or an auditory disorder. In some embodiments, the neurological condition is pain (e.g. chronic pain). In some embodiments, the neurological condition is depression, bipolar disorder, anxiety, social anxiety, post-traumatic stress disorder (PTSD), panic disorder, phobia, schizophrenia, psychopathy, or antisocial personality disorder. In some embodiments, the neurological condition is an impulsive disorder. In some embodiments, the impulsive disorder is attention deficit hyperactivity disorder (ADHD), attention deficit disorder (ADD), Tourette's syndrome or autism. In some embodiments, the neurological condition is a compulsive disorder. In some embodiments, the compulsive disorder is obsessive compulsive disorder (OCD), gambling, or aberrant sexual behavior. In some embodiments, the neurological condition is a personality disorder. In some embodiments, the personality disorder is conduct disorder, antisocial personality, or aggressive behavior. In some embodiments, the neurological condition is an involuntary condition. In some embodiments, the involuntary condition comprises involuntary behaviors such as nervous myoclonus or twitching (e.g., facial twitching), hiccups, etc.

In any of the embodiments provided herein, the disorders or conditions are neurological disorders or conditions. In some embodiments, the disorders or conditions are neurocognitive disorders or conditions. In some embodiments, the disorders or conditions are neurodegenerative disorders or conditions. In some embodiments, the symptoms of the neurological condition are physical, behavioral, emotional, mental, or a combination thereof.

In any of the embodiments provided herein, the methods may improve a user's ability to cope with or overcome disorders, conditions or symptoms, which may include but not be limited to addiction disorders, such as but not limited to alcohol abuse, substance abuse, smoking, or obesity. In any of the embodiments provided herein, the methods may improve a user's ability to cope with or overcome disorders, conditions or symptoms including but not limited to eating disorders and auditory disorders. In any of the embodiments provided herein, the methods may improve a user's ability to cope with or overcome disorders, conditions or symptoms including but not limited to pain, such as but not limited to chronic pain. In any of the embodiments provided herein, the methods may improve a user's ability to cope with or overcome disorders, conditions or symptoms including but not limited to depression, bipolar disorder, post-traumatic stress disorder (PTSD), panic disorder, phobia, schizophrenia, psychopathy, or antisocial personality disorder. In any of the embodiments provided herein, the methods may improve a user's ability to cope with or overcome disorders, conditions or symptoms including but not limited to impulse disorders, such as but not limited to attention deficit hyperactivity disorder (ADHD), Tourette's syndrome or autism. In any of the embodiments provided herein, the methods may improve a user's ability to cope with or overcome disorders, conditions or symptoms including but not limited to compulsive disorder, such as but not limited to obsessive compulsive disorder (OCD), gambling, or aberrant sexual behavior. In any of the embodiments provided herein, the methods may improve a user's ability to cope with or overcome disorders, conditions or symptoms including but not limited to personality disorders, such as but not limited to conduct disorder, antisocial personality, or aggressive behavior. In some embodiments, the neurological condition is an involuntary condition. In some embodiments, the involuntary condition comprises involuntary behaviors such as nervous myoclonus or twitching (e.g., facial twitching), hiccups, etc.

In any of the embodiments provided herein, the reference population data, the historical user data, or a combination thereof, are gathered over a time period. In any of the embodiments provided herein, the time period comprises at least 1 day (e.g., 2, 3, 4, 5, 6, or 7 days). In any of the embodiments provided herein, the time period comprises at least 1 week (e.g., 2, 3, or 4 weeks). In any of the embodiments provided herein, the time period comprises at least 1 month (e.g., 2, 3, 4, 5, 6, 7, 8, 9, 10, 11, or 12 months). In any of the embodiments provided herein, the time period comprises at least 1 year (e.g., 2, 3, 4, 5, 6, 7, 8, 9, 10, 11, or 12 years). In any of the embodiments provided herein, the reference population data, the historical user database, or a combination thereof, are received from a third party library. In any of the embodiments provided herein, the reference population data is gathered by: (i) receiving data from one or more data sources associated with a reference population, and (ii) processing the data received from the one or more data sources. In any of the embodiments provided herein, the historical user database is gathered over the time period according to the method of claim 1. For example, the historical user database can be gathered by receiving data from one or more data sources. Gathering the historical user database may also comprise processing the data received from the one or more data sources to generate or extract one or more user specific parameters from the received data. Gathering the historical user database may also comprise initializing one or more models of well-being incorporating data from at least a reference population or a historical user database, and generating an output indicative of a state of the user's well-being based at least in part on the user specific parameters and the data from at least the reference population or the historical user database using the one or more models.

In any of the embodiments provided herein, the method may further comprise: updating the one or more models based at least in part on newly received data from: (i) the one or more data sources, (ii) the historical user database, or (iii) the reference population. In any of the embodiments provided herein, wherein updating the one or more models comprises updating the one or more user specific parameters based at least in part on the newly received data.

In any of the embodiments provided herein, the method may further comprise generating one or more recommendations based at least in part on the output. In any of the embodiments provided herein, the one or more recommendations comprises providing a customized treatment regimen using one or more drugs to be taken by the user. In any of the embodiments provided herein, the one or more drugs comprises a 5-HT receptor agonist, a prescribed medication, an over the counter drug, a dietary supplement, a plant-derivative substance, a natural product or a combination thereof. In any of the embodiments provided herein, the customized treatment regimen comprises a dose modification. In any of the embodiments provided herein, the dose modification comprises a recommendation to increase or decrease a dose amount, a dose frequency, or a combination thereof, of at least one of the one or more drugs. In any of the embodiments provided herein, the one or more recommendations comprises one or more behavior modifications. In any of the embodiments provided herein, the one or more behavior modifications comprises a modification to: substance intake, physical exercise, diet, sleep schedule, cognitive behaviors, self-defeating behaviors, social interactions, compulsive behaviors, addictions, or a combination thereof. In any of the embodiments provided herein, the one or more recommendations comprises providing a combination of a customized dosing regimen and one or more behavior modifications.

In any of the embodiments provided herein, the method may further comprise providing a predicted efficacy associated with each of the one or more recommendations. In any of the embodiments provided herein, the method may further comprise generating a ranking for each of the one or more recommendations. In any of the embodiments provided herein, the predicted efficacy is provided based on a probability density function. In any of the embodiments provided herein, the one or more recommendations are recommended if the one or more recommendations reach a threshold level of predicted efficacy. In any of the embodiments provided herein, the ranking is based at least in part on the predicted efficacy.

In any of the embodiments provided herein, the method may further comprise displaying the one or more recommendations to the user on a graphical user interface (e.g., of a user device). In any of the embodiments provided herein, the graphical user interface comprises a verbal recommendation. In any of the embodiments provided herein, the graphical user interface comprises a visual representation of the one or more recommendations. In any of the embodiments provided herein, the visual representation comprises a representation of a human body comprising one or more highlighted body parts associated with the user's well-being. In any of the embodiments provided herein, the method may further comprise providing the one or more recommendations to one or more third parties (e.g., a doctor, a therapist, fitness coach, wellness coach, lifestyle coach, spiritual guide, religious guide, a healthcare worker, a clinician, a teacher or professor, a researcher (e.g., field expert), or a combination thereof) associated with the user. In any of the embodiments provided herein, the method may further comprise (i) receiving third party input to the one or more recommendations, and (ii) updating the one or more recommendations based at least in part on the third party input.

In any of the embodiments provided herein, the one or more data sources comprises a plurality of data sources. In any of the embodiments provided herein, the one or more data sources each being independently selected from the group consisting of user input, third party input, device input, and sample input.

In any of the embodiments provided herein, the user input comprises data received from one or more user devices (e.g., a mobile device, tablet, or other personal computing device, or any or any user connected device (e.g., internet of things device (e.g., associated with the user's environment or daily living (e.g., smart thermostat, smart device, etc.))). In any of the embodiments provided herein, receiving the data comprises administering one or more surveys or questionnaires to the user. In any of the embodiments provided herein, the user input comprises user responses to the one or more surveys or questionnaires. In any of the embodiments provided herein, the user input comprises quantitative, qualitative, or a combination thereof, input. In any of the embodiments provided herein, the one or more surveys or questionnaires are administered at least once (e.g., twice, three times, four times, five times, six times, seven times, eight times, nine times, or ten times) daily to the user. In any of the embodiments provided herein, the one or more surveys or questionnaires are administered at least once (e.g., twice, three times, four times, five times, six times, seven times, eight times, nine times, or ten times) a week to the user. In any of the embodiments provided herein, the one or more surveys or questionnaires are administered at least once (e.g., twice, three times, four times, five times, six times, seven times, eight times, nine times, or ten times) a month to the user. In any of the embodiments provided herein, the one or more surveys or questionnaires comprises surveys acquiring emotional data of the user. In any of the embodiments provided herein, the emotional data comprises data relating to depression, anxiety, stress, coping, mood, sleep, attention, quality of life, phobias, demoralization, rumination, social interaction (e.g., a quality or a quantity thereof), or a combination thereof, of the individual. In any of the embodiments provided herein, the one or more surveys or questionnaires comprises surveys acquiring physical data of the user. In any of the embodiments provided herein, the physical data comprises data relating to the user's physiological state (e.g., weight, height, diet, arousal, sleep, dreams), physical exercise (e.g., amount of physical exercise), substance consumption, or a combination thereof. In any of the embodiments provided herein, the one or more surveys or questionnaires comprises surveys acquiring data of one or more stressors of the user. In any of the embodiments provided herein, the one or more stressors comprises data relating to the user's daily interactions (e.g., commute, number of meetings at work, exams in school, etc.). In any of the embodiments provided herein, the one or more stressors comprises data relating to long term stress comprising stressors associated with family, social life, financial status, health, accidents, response to news, or a combination thereof. In any of the embodiments provided herein, the one or more surveys or questionnaires (e.g., written or verbal) comprises a Beck Depression Inventory (BDI), a Generalized Anxiety Disorder (GAD-7), a Depression Anxiety Stress Scale (DASS), a Brief-COPE, a Positive and Negative Affect Schedule (PANAS), a State Trait Anxiety Inventory (STAI), a modified version of a Russel Mood Circumplex, a modified version of a NIH Sleep Diary, a 36 Item Short Form Health Survey (SF-36), a 5D Altered state of Consciousness Scale (5d-ASC), a daily sleep diary questionnaire, a Hamilton Rating Scale for Depression, a Hamilton Anxiety Rating Scale, mood surveys, self-reporting surveys (e.g., with questions about attention, social interactions, fatigue), sleep diaries, or a combination thereof. In any of the embodiments provided herein, the user input comprises the user's unprompted input related to the user's well-being (e.g., input relating to the user's social interactions, emotional state, physical state, one or more stressors, input to a user diary, or a combination thereof). In any of the embodiments provided herein, the unprompted input comprises quantitative, qualitative, or a combination thereof, input. In any of the embodiments provided herein, the user's unprompted input comprises a self-examination of the user's well-being.

In any of the embodiments provided herein, the third party input comprises data from the user's doctor(s), family, friends, co-workers, therapists, counselors, teachers, professors, spiritual guides, religious guides, religious guides, fitness coach, wellness coach, lifestyle coach, healthcare workers, or a combination thereof. In any of the embodiments provided herein, the third party input comprises doctor input (e.g., observations (e.g., after a doctor-user visit)) on the user's well-being. In any of the embodiments provided herein, the third party input comprises input (e.g., observations) from the user's family, friends, co-workers, therapists, counselors teachers, professors, spiritual guides, religious guides, religious guides, fitness coach, wellness coach, lifestyle coach, healthcare workers, or a combination thereof.

In any of the embodiments provided herein, the device input comprises data from one or more user devices. In any of the embodiments provided herein, the one or more user devices comprises physiological, neurological, psychological, metabolic, or biological data of the user. In any of the embodiments provided herein, the one or more user devices comprises: one or more personal computing devices (e.g., a mobile device, tablet, or other personal computing device), one or more applications associated with the one or more personal computing devices (e.g., social media applications (e.g., Instagram, Facebook, Twitter, TikTok, Google Maps, Waze, Calendar, Microphone, Online Purchasing apps), one or more wearable devices (e.g., comprising one or more sensors for measuring a physiological state of the user (e.g., which may be connected to the one or more personal computing devices)), one or more implantable devices, one or more user connected devices, or a combination thereof. In any of the embodiments provided herein, receiving the data comprises performing an evaluation (e.g., a physiological (e.g., a brain) evaluation) on the user using the one or more wearable devices, or the one or more implanted devices. In any of the embodiments provided herein, the evaluation is performed at least once (e.g., twice, three times, four times, five times, six times, seven times, eight times, nine times, or ten times) daily to the user. In any of the embodiments provided herein, the evaluation is performed at least once (e.g., twice, three times, four times, five times, six times, seven times, eight times, nine times, or ten times) a week to the user. In any of the embodiments provided herein, the evaluation is performed at least once (e.g., twice, three times, four times, five times, six times, seven times, eight times, nine times, or ten times) a month to the user. In any of the embodiments provided herein, the one or more wearable devices measures: a brain activity (e.g. using a brain evaluation (e.g. electroencephalogram (EEG), magnetoencephalogram (MEG), functional near-infrared spectroscopy (fNRIS), Positron Emission Tomography (PET) transcranial functional ultrasound)), a heart test (e.g. evaluated using an EEG for recording heart rate or electrocardiogram (EKG) for recording heart rhythm), a visual test, an auditory test, functional data (e.g. evaluated by brain imaging (e.g. fNRIS, PET) or monitoring of brain activity levels (e.g. EEG)), body temperature, food intake, metabolic rate, perspiration, hydration, salivation, pupil dilation, breathing rate, pulse rate, skin color, or skin temperature, and the like, of the user.

In any of the embodiments provided herein, the one or more wearable devices measures physiological, neurological, psychological, metabolic, or biological activity or a combination thereof of the user. In some embodiments the activity of a user is measured or identified by a brain activity (e.g. using a brain evaluation (e.g. EEG, MEG, fNRIS, PET transcranial functional ultrasound)), a heart test (e.g. evaluated using an EEG for recording heart rate or EKG for recording heart rhythm), a visual test, an auditory test, a biological sample (e.g. for evaluating changes in serum, plasma, whole blood, urine, sweat or the like), patient reporting (e.g. through questionnaires or surveys to evaluate mood, affect, coping, sleep quality, stress, anxiety, memory, and/or other emotions), functional data (e.g. evaluated by brain imaging (e.g. fNRIS, PET) or monitoring of brain activity levels (e.g. EEG)), body temperature, food intake, metabolic rate, perspiration, hydration, salivation, pupil dilation, breathing rate, pulse rate, skin color, or skin temperature, and the like. In any of the embodiments provided herein, the one or more wearable devices comprises an electroencephalogram (EEG) device (e.g., multi-electrode EEG device). In any of the embodiments provided herein, the EEG device comprises two or more electrodes that make contact with the forehead of the user. In any of the embodiments provided herein, the EEG device makes contact with the user's forehead for at least thirty seconds (e.g., at least 1, 2, 3, 4, 5, 6, 7, 8, 9, 10, 15, 20, 25, 30, 35, 40, 45, 50, 55, or 60 minutes). In any of the embodiments provided herein, the one or more wearable devices comprises one or more smart devices (e.g., smartwatches) worn by the user configured to measure physiological, neurological, psychological, metabolic, or biological data of the user.

In some embodiments, the activity of a user may be measured or identified by evaluating one or more of EEG, EKG, MEG, fNIRS, PET, transcranial functional ultrasound, visual stimuli (e.g. colour, shape, pattern, emotional face, video, flash, milli second or longer), auditory stimuli (e.g. matched paired, acoustic frequency, Hz, milli second or longer), ultrasound waves by a sensor e.g., a piezoelectric resonant material (e.g., a PZT, a CMUT, a PMUT), clinical effect (visual, auditory, body, time and space, cognition, drowsiness, confusion, impairment), standard/deviant waveforms, evoked power/potential, voltage (alpha bands, beta bands, gamma bands, delta bands, or theta bands), asynchronization, time-locked, magnetic, hemodynamic (flux, flow, velocity, oxygenation), MMN, ASSR, surveys or questionnaires (written or verbal) including 5D-ASC, Beck Depression, Coping, DASS42, Gad-7 Anxiety, PANAS-GEN, SF-36 QOL, STAI 6, brain imaging tests, heart rate, cardiovascular activity, photodiode, skin conduction and impedance, biological samples such as serum, plasma, whole blood, urine, sweat or the like, visual perception alteration, an auditory perception alteration, bodily perception alteration, a temporal perception alteration, or a spatial perception alteration, sleep quality, patient reported outcomes (e.g. electronic) or subjective mood, affect, coping, sleep quality, stress, anxiety, memory, and other emotional or functional data with brain imaging or brain activity, providing data (e.g., patterns, clusters, classifications, amplitudes, frequencies, or magnitude) patient evaluation (e.g., physicians, counselors, psychologist, or spiritual leader), data comprising visual representations of brain responses or brain activity data and/or survey or patient reported outcomes to help convey the effectiveness or lack thereof of a psychedelic therapy, training modules, machine learning algorithms and training sets, artificial intelligence (e.g., deep convolution neural networks) algorithms and/or machine learning processes to classify, cluster, or recognize patterns or relationships amongst sensory-evoked (e.g., auditory or visual stimuli) brain activity or resting state brain activity acquired by brain imaging methods and patient reported outcomes (e.g., changes in mood, anxiety, motivation, or memory), auditory brainstem responses (ABR), paired pulse inhibition (PPI; ie P50 auditory suppression; sensory gating), auditory mismatch negativity (MNN), and auditory steady state responses (ASSR), if EEG measures of auditory sensory evoked activity, cognitive control of attention and emotion, sub-thalamic and cortical levels, amplitudes of MMN potentials will be correlated to depression, anxiety, and mood survey data, Emotional Flanker Task, Erikson Flanker Task, Continuous Performance Test (CPT), Connor's CPT, oddball tasks, State Trait Anxiety Inventory is a 6-item, self-report survey, sleep diaries, the symmetry and power of alpha, beta, delta, and gamma brainwave activity across prefrontal cortex brain regions, Medical Quality of Life Outcomes Study 36-Item Short Form Health Survey (SF-36) and Physical Component Summary (PCS) and Mental Component Summary (MCS) scores, physical functioning, role physical, role emotional, bodily pain, vitality, social functioning, mental health, and general health on Likert, salivary cytokines, salivary brain-derived neurotrophic factors, psychophysical testing, assessing congruent, incongruent, and neutral conditions, surveys assessing mood, depression, anxiety, and stress, Mood Circumplex, DSM-V, cognitive behavioral therapy, synergy score, serenity score, quality of life, Sleep diary and Mood Capture, questions about attention, social interactions, fatigue and mood, approach avoid metric, biometric of coping, Heart Rate Variability (HRV) spot check, or camera.

In any of the embodiments provided herein, the sample input comprises data from one or more biological samples. In any of the embodiments provided herein, the one or more biological samples comprises user serum, plasma, tissue, whole blood, urine, fecal samples, sweat, or saliva. In any of the embodiments provided herein, data from one or more user biological samples comprises user blood glucose levels, blood alcohol levels, plasma drug (e.g., a 5-HT receptor agonist, medication, or over the counter drug) concentration levels, hormone levels, metabolomics, genetic information, or combinations thereof. In any of the embodiments provided herein, the one or more biological samples are obtained from a laboratory (e.g., clinical, hospital, bio bank, or outpatient laboratory), the user (e.g., at home sampling (e.g., of saliva, urine, fecal matter)), or a combination thereof. In any of the embodiments provided herein, the sample input comprises data related to one or more biomarkers of the user. In any of the embodiments provided herein, the data related to the one or more biomarkers of the user is identified from the one or more biological samples. In any of the embodiments provided herein, the one or more biomarkers are indicative of responsiveness to a treatment regimen. In any of the embodiments provided herein, the one or more biomarkers comprises inflammatory biomarkers (e.g., interleukins or cytokines), neurotransmitter metabolites, or a combination thereof. In any of the embodiments provided herein, the one or more biomarkers comprises diagnostic, monitoring, pharmacodynamic/response, predictive, prognostic, safety, and/or susceptibility/risk biomarkers.

In any of the embodiments provided herein, processing the data is performed using one or more data processing algorithms. In any of the embodiments provided herein, the one or more data processing algorithms comprises one or more feature extraction algorithms, one or more machine learning algorithms, one or more artificial intelligence algorithms, one or more Bayesian algorithms (e.g., Bayesian assimilation), one or more statistical analysis algorithms, or a combination thereof. In any of the embodiments provided herein, processing the data is performed in a real-time, near real-time, or dynamic nature. In any of the embodiments provided herein, processing the data comprises batch processing. In any of the embodiments provided herein, the one or more data processing algorithms receive data from: (i) the one or more data sources, (ii) a database, or a combination thereof. In any of the embodiments provided herein, the database comprises stored reference population data, stored historical user specific data, or a combination thereof. In any of the embodiments provided herein, the one or more data processing algorithms comprise a natural language processing model configured to extract qualitative data from the one or more data sources, the historical user database, a reference population, or a combination thereof. In any of the embodiments provided herein, processing the received data further comprises processing the data from the historical user database, the reference population, or a combination thereof. In any of the embodiments provided herein, processing the data further comprises generating or extracting one or more labels from the reference population data. In any of the embodiments provided herein, processing the data further comprises identifying arbitrary data, data outliers (e.g., missing data or data falling outside a trend), or a combination thereof. In any of the embodiments provided herein, the processing further comprises filling in missing data using one or more data interpolation methods. In any of the embodiments provided herein, updating the one or more models comprises updating the one or more labels based at least in part on the newly received data. In any of the embodiments provided herein, the one or more user specific parameters comprises data associated with: drug use (e.g., a 5-HT receptor agonist, medication, over the counter drug, a dietary supplement, a plant-derivative substance, a natural product, a performance enhancer or a combination thereof) data, association with third parties (e.g., a doctor or therapist) data, emotional data, physical data, social interaction data, data related to one or more stressors, physiological data, neurological data, psychological data, metabolic data, or biological data. In any of the embodiments provided herein, the one or more labels identifies data in reference population associated with: drug use (e.g., a 5-HT receptor agonist, medication, over the counter drug, or a combination thereof) data, association with third parties (e.g., a doctor or therapist) data, emotional data, physical data, social interaction data, data related to one or more stressors, physiological data, neurological data, psychological data, metabolic data, or biological data.

In any of the embodiments provided herein, the one or more models comprises one or more pre-programmed models. In any of the embodiments provided herein, the one or more models comprises one or more artificial intelligence models. In any of the embodiments provided herein, the one or more artificial intelligence models comprises one or more neuromorphic computing models. In any of the embodiments provided herein, the one or more neuromorphic computing models comprises a neural network (e.g., a spiking neural network). In any of the embodiments provided herein, the one or more models comprises one or more machine learning models. In any of the embodiments provided herein, the one or more machine learning models comprises one or more artificial intelligence models. In any of the embodiments provided herein, the one or more machine learning models comprises a neural network (e.g., a spiking neural network, a deep neural network, a dynamic neural network, or a convolutional neural network), a regression-based learning algorithm, a linear or non-linear algorithm, a feed-forward neural network, a generative adversarial network (GAN), deep residual networks, a genetic algorithm, or any combination thereof. In any of the embodiments provided herein, the one or more machine learning models comprises trained machine learning models. In any of the embodiments provided herein, the one or more machine learning models comprises supervised machine learning models, unsupervised machine learning models, or a combination thereof. In any of the embodiments provided herein, the one or more machine learning models compares the data received from the one or more data sources to the historical user database, the reference population data, or a combination thereof. In any of the embodiments provided herein, the one or more models comprises, one or more pre-programmed models, one or more artificial intelligence models, one or more machine learning models, or a combination thereof. In any of the embodiments provided herein, the one or more machine learning models generates an association between the user and the reference population data based at least in part on the one or more user specific parameters and the one or more labels. In any of the embodiments provided herein, the one or more machine learning models compares the data received from the one or more data sources to the reference population data using at least the association generated between the user and the reference population. In any of the embodiments provided herein, the output is generated based at least in part on the comparison of the data received from the one or more data sources to the reference population data. In any of the embodiments provided herein, the output is generated based at least in part on the comparison of the data received from the one or more data sources to historical user data stored on the database. In any of the embodiments provided herein, the output is generated in real-time, near real-time, or in a dynamic nature.

In any of the embodiments provided herein, the output is a score. In any of the embodiments provided herein, the score is a qualitative score, a quantitative score, or a combination thereof. In any of the embodiments provided herein, the score is positive, negative, or neutral. In any of the embodiments provided herein, the positive score is extremely positive, very positive, moderately positive, or slightly positive. In any of the embodiments provided herein, the negative score is extremely negative, very negative, moderately negative, or slightly negative. In any of the embodiments provided herein, the score is a number on a scale from 0-100, wherein 0 indicates extremely negative, 50 indicates neutral, and 100 indicates extremely positive.

In any of the embodiments provided herein, the one or more recommendations is generated in real-time, near real-time, or in a dynamic nature. In any of the embodiments provided herein, the one or more recommendations is displayed to the user on a graphical user interface (e.g., of a user device) in real-time, near real-time, or in a dynamic nature. In any of the embodiments provided herein, the one or more recommendations is provided to the one or more third parties (e.g., a doctor, a therapist, or a combination thereof) associated with the user in real-time, near real-time, or in a dynamic nature. In any of the embodiments provided herein, the one or more recommendations is generated in less than 5 minutes (e.g., 4, 3, 2, or 1 minutes) from the time of receiving the data from the one or more data sources. In any of the embodiments provided herein, the one or more recommendations is displayed to the user, or provided to the third party, or both, in less than 5 minutes (e.g., 4, 3, 2, or 1 minutes) from the time of receiving the data from the one or more data sources.

In any of the embodiments provided herein, the method may further comprise simulating the user's well-being based at least in part on the user specific parameters and the data from the one or more data sources, the historical user database, or from the reference population. In any of the embodiments provided herein, wherein the generating the output is further based at least in part on the simulation of the user's well-being. In any of the embodiments provided herein, the method may further comprise monitoring the user's well-being over a period of time. In any of the embodiments provided herein, the monitoring comprising continuous monitoring or discrete monitoring. In any of the embodiments provided herein, the period of time comprises at least 1 day (e.g., 2, 3, 4, 5, 6, or 7 days). In any of the embodiments provided herein, the period of time comprises at least 1 week (e.g., 2, 3, or 4 weeks). In any of the embodiments provided herein, the period of time comprises at least 1 month (e.g., 2, 3, 4, 5, 6, 7, 8, 9, 10, 11, or 12 months). In any of the embodiments provided herein, the period of time comprises at least 1 year (e.g., 2, 3, 4, 5, 6, 7, 8, 9, 10, 11, or 12 years). In any of the embodiments provided herein, the one or more recommendations is further based at least in part on a progress, digression, or combination thereof, of the user's well-being over the monitored period of time.

Provided in some embodiments here is a method for monitoring the effectiveness of a 5-hydroxytryptamine (5-HT) receptor agonist (e.g., psilocybin), comprising measuring (e.g., on a network (e.g., WiFi, cloud, BLE, 3/4/5G)) one or more brain response using a node or a wearable brain imaging device (e.g., an EEG), wherein the patient listens to paired or sustained auditory stimuli (e.g., at home, at work, or at school), the auditory stimuli being delivered from an application on a (e.g., mobile) device (e.g., cellular phone, tablet, or the like).

Provided in some embodiments herein is a method for using Bayesian statistics and methods to estimate and predict confidence in the effectiveness of a psychedelic treatment or dose based on historical brain imaging data in a database for particular patient types.

Provided in some embodiments herein is a method of imaging brain activity (e.g., voltage) in an individual using a wearable device (e.g., EEG (e.g., multi-electrode EEG device)), the wearable device having two or more electrodes that make contact with the forehead of the individual for at least thirty seconds and up to twenty minutes.

Provided in some embodiments herein is a method for optically imaging brain activity in an individual (e.g., from a wearable device) to determine, track, or optimize the effectiveness of a psychedelic treatment, wherein a brain signal is identified by transmission of visible light (400-680 nanometers) or infrared light (greater than or equal to 680 nanometers) across the skin into the brain to collect reflected photons by a sensor (e.g., a photodiode or CMOS).

Provided in some embodiments is a method of acoustically imaging brain activity in an individual (e.g., from a wearable device) to determine, track, or optimize the effectiveness of a psychedelic treatment, wherein a brain signal is identified by transmission of ultrasound (e.g., having an acoustic frequency of greater than or equal to one megahertz) (e.g., across the skin and skull) into the brain to collect reflected ultrasound waves by a sensor (e.g., a piezoelectric resonant material (e.g., a PZT, a CMUT, a PMUT).

In some embodiments, the method comprises using artificial intelligence (e.g., deep convolution neural networks) algorithms and/or machine learning processes to classify, cluster, or recognize patterns or relationships amongst sensory-evoked (e.g., auditory or visual stimuli) brain activity or resting state brain activity acquired by brain imaging methods and patient reported outcomes (e.g., changes in mood, anxiety, motivation, or memory) for optimizing the dosing schedule or treatment paradigm or a patient undergoing treatment with psychedelic substances (e.g. psilocybin).

Provided in some embodiments herein is a computer-implemented method for identifying an effectiveness of a psychedelic treatment administered to an individual.

Provided in some embodiments herein is a computer-implemented method for identifying a therapeutically effective dose of a 5-HT receptor agonist administered to an individual. In some embodiments, the method comprises administering the 5-HT receptor agonist to the individual. In some embodiments, the method comprises emitting one or more (e.g., auditory or visual) to the individual. In some embodiments, the method comprises receiving, from a brain imaging device (e.g., an EEG), a brain response. In some embodiments, the method comprises identifying the therapeutically effective dose of the 5-HT receptor agonist based at least in part on the one or more (e.g., auditory or visual) stimulus and the brain response.

In some embodiments, the method further comprises receiving, from the individual, an emotional data. In some embodiments, the therapeutically effective dose of the 5-HT receptor agonist is further determined based on the emotional data. In some embodiments, the emotional data comprises a mood rating, a sleep rating, a stress rating, an anxiety rating, a memory rating, or any combination thereof.

In some embodiments, the method comprises transmitting (e.g., the effectiveness of) the therapeutically effective dose of the 5-HT receptor agonist to the individual, a caregiver, or both.

In some embodiments, two or more of any step provided herein are performed simultaneously.

In some embodiments, two or more of any step provided herein are performed sequentially.

In some embodiments, (e.g., the effectiveness of) the therapeutically effective dose of the 5-HT receptor agonist is transmitted by a mobile device (e.g., cellular phone, tablet). In some embodiments, (e.g., the effectiveness of) the therapeutically effective dose of the 5-HT receptor agonist is transmitted over a wireless network (e.g., WiFi, cloud, BLE, 3/4/5G).

In some embodiments, (e.g., the effectiveness of) the therapeutically effective dose of the 5-HT receptor agonist is determined by a machine learning algorithm.

Provided in some embodiments herein is a computer-implemented system for identifying an effectiveness of a psychedelic treatment administered to an individual.

Provided in some embodiments herein is a computer-implemented system for identifying a therapeutically effective dose of a 5-HT receptor agonist administered to an individual.

In some embodiments, the system comprises a digital processing device. In some embodiments, the digital processing device comprises at least one processor. In some embodiments, the digital processing device comprises an operating system configured to perform executable instructions. In some embodiments, the digital processing device comprises a memory. In some embodiments, the digital processing device comprises a computer program including instructions executable by the digital processing device to create an application.

Provided in some embodiment herein is a non-transitory computer-readable storage media encoded with a computer program including instructions executable by a processor to create an application for identifying an effectiveness of a psychedelic treatment administered to an individual.

Provided in some embodiment herein is a non-transitory computer-readable storage media encoded with a computer program including instructions executable by a processor to create an application for identifying a therapeutically effective dose of a 5-HT receptor agonist administered to an individual.

In some embodiments, the application is configured to receive an indicator of the 5-HT receptor agonist administered to the 5-HT receptor agonist provided to the individual. In some embodiments, the application is configured to direct a device (e.g., an auditory or visual device) to emit one or more (auditory or visual) stimulus to the individual. In some embodiments, the application is configured to receive, from a brain imaging device (e.g., EEG), a brain response. In some embodiments, the application is configured to identify the therapeutically effective dose of the 5-HT receptor agonist based at least in part on the one or more (auditory or visual) stimulus and the brain response.

In some embodiments, the application is further configured to receive, from the individual, an emotional data. In some embodiments, the application is further configured to determine the therapeutically effective dose of the 5-HT receptor agonist based on the emotional data.

In some embodiments, the emotional data comprises a mood rating, a sleep rating, a stress rating, an anxiety rating, a memory rating, or any combination thereof.

In some embodiments, the application is configured to simultaneously perform two or more of steps provided herein.

In some embodiments, the application is configured to sequentially perform two or more of steps provided herein.

In some embodiments, the application is further configured to transmit the therapeutically effective dose of the 5-HT receptor agonist to the individual, a caregiver, or both. In some embodiments, the application directs the transmission of the therapeutically effective dose of the 5-HT receptor agonist by a mobile device (e.g., cellular phone, tablet). In some embodiments, the application directs the transmission of the therapeutically effective dose of the 5-HT receptor agonist over a wireless network (e.g., WiFi, cloud, BLE, 3/4/5G). In some embodiments, the therapeutically effective dose of the 5-HT receptor agonist is determined by a machine learning algorithm.

Provided in some embodiments herein is a computer-implemented system for identifying an effectiveness of a psychedelic treatment administered to an individual.

Provided in some embodiments herein is a computer-implemented system for identifying a therapeutically effective dose of a 5-HT receptor agonist administered to an individual. In some embodiments, the system comprises a digital processing device. In some embodiments, the digital processing device comprises at least one processor. In some embodiments, the digital processing device comprises an operating system configured to perform executable instructions. In some embodiments, the digital processing device comprises a memory. In some embodiments, the digital processing device comprises a computer program including instructions executable by the digital processing device to create an application.

Provided in some embodiments is a non-transitory computer-readable storage media encoded with a computer program including instructions executable by a processor to create an application for identifying an effectiveness of a psychedelic treatment administered to an individual.

Provided in some embodiments is a non-transitory computer-readable storage media encoded with a computer program including instructions executable by a processor to create an application for identifying a therapeutically effective dose of a 5-HT receptor agonist administered to an individual.

In some embodiments, the application is configured to apply a first machine learning algorithm to a plurality of 5-HT receptor agonist doses (e.g., dose responses), a plurality of (e.g., auditory or visual) stimuli, and brain responses to identifying the therapeutically effective dose of the 5-HT receptor agonist.

In some embodiments, the application is configured to receive verified emotional data regarding an emotional effectiveness of the 5-HT receptor agonist.

In some embodiments, the application is configured to feed back the verified data to improve the first machine learning algorithm's calculation over time.

In some embodiments, the first machine learning algorithm is trained by a neural network. In some embodiments, the neural network comprises a first training module for creating a first training set comprising a set of 5-HT receptor agonist doses (e.g., dose responses), each dose associated with one (e.g., auditory or visual) stimulus, and one brain response. In some embodiments, the neural network comprises a first training module training the neural network using the first training set. In some embodiments, the neural network comprises a second training module creating a second training set for second stage training comprising the first training set and the psychedelic treatments incorrectly detected as having a positive effectiveness on the individual after the first stage of training. In some embodiments, the neural network comprises training the neural network using the second training set.

In some embodiments, the application is configured to perform administering (e.g., at least once weekly for 5 or more weeks) one or more surveys or questionnaires to the individual to acquire emotional data. In some embodiments, questionnaires are administered once, twice, three times, four times, five times, six times, seven times, eight times, nine times, ten times, eleven times, twelve times, thirteen times, or fourteen times of more weekly for at least 1, 2, 3, 4, 5, 6, 7, 8, 9, 10, 11, or 12 or more weeks, or for 4, 5, 6, 7, 8, 9, 10, 11, 12 or more months, or for 1, 2, 3, or 4 or more years.

In some embodiments, the emotional data comprises data regarding depression, anxiety, stress, coping, mood, sleep, and quality of life of the individual.

In some embodiments, the one or more surveys or questionnaires can include a Beck Depression Inventory (BDI), a Generalized Anxiety Disorder (GAD-7), a Depression Anxiety Stress Scale (DASS), a Brief-COPE, a Positive and Negative Affect Schedule (PANAS), a State Trait Anxiety Inventory (STAI), a modified version of a Russel Mood Circumplex, a modified version of a NIH Sleep Diary, a 36 Item Short Form Health Survey (SF-36), a 5D Altered state of Consciousness Scale (5d-ASC), and/or a daily sleep diary questionnaire.

In some embodiments, the application is configured to perform receiving (e.g., at least once weekly for 5 or more weeks) an individual's responses to the one or more surveys or questionnaires.

In some embodiments, the application is configured to perform scoring the individual's responses to the one or more surveys or questionnaires.

In some embodiments, identifying the therapeutically effective dose of the 5-HT receptor agonist is based at least in part on the scoring of the one or more surveys or questionnaires.

In some embodiments, the application is configured to perform receiving resting state brain activity from the individual. In some embodiments, the resting state brain activity is measured for at least thirty seconds (e.g., at least once, and up to four times, daily). In some embodiments, identifying the therapeutically effective dose of the 5-HT receptor agonist is based at least in part on changes to the resting state brain activity.

In some embodiments, receiving, from a brain imaging device (e.g., EEG), a brain response, comprises receiving amplitude and spectral power of EEG potentials measured from frontal, temporal, and parietal EEG sites in response to the one or more auditory and/or visual stimulus. In some embodiments, the one or more auditory stimulus comprises one or more auditory tasks comprising P50 paired click auditory suppression, Mismatch Negativity (MMN), and/or Auditory Steady State Response (ASSR). In some embodiments, the one of more auditory tasks is administered to the individual 1.5 hours after the 5-HT receptor agonist is administered to the individual. In some embodiments, the one or more visual stimulus comprises one or more visual tasks comprising an Emotional Flanker Task and/or a Continuous Performance Test (CPT). In some embodiments, identifying the therapeutically effective dose of the 5-HT receptor agonist is based at least in part on the brain response received to the one or more auditory tasks and/or the one or more visual tasks.

In some embodiments, identifying the therapeutically effective dose of the 5-HT receptor agonist is performed using one or more statistical methods comprising Bayesian methods, Mixed Model Repeated Measures (MMRM), repeated measures ANOVA and ANCOVA, and regression analyses.

Provided in some embodiments herein is a computer-implemented method for affecting a user's well-being, comprising: (a) receiving data from one or more data sources, wherein receiving the data comprises receiving user input from one or more surveys or questionnaires, performing an evaluation on the user using one or more wearable devices and recording the data gathered from the evaluation, or a combination thereof; (b) analyzing the data received from the one or more data source; and (c) displaying a visual representation of a state of the user's well-being to the user based at least in part on the analyzing in (b). In some embodiments, the visual representation comprises an image of the user's brain activity (e.g., frequency band activity). In some embodiments, the data gathered from the evaluation is stored in the cloud twice daily (e.g., morning and evening).

The systems and methods described above provide several improvements to the field of affecting, improving, simulating, and monitoring a user's well-being. As non-limiting examples, the systems can receive and process data of a user to identify user specific parameters associated with the user's well-being. The systems can initialize one or more models incorporating data from a reference population or historical user database and generate an output using the user specific parameters that is indicative of a state of the user's well-being. The systems can generate user-specific treatment regimens, behavior modifications, and other recommendations to affect, monitor, and improve the user's physical, mental, emotional, social, etc., well-being. The systems can provide an associated efficacy for the one or more recommendations and rank the one or more recommendations based on their applicability to a certain user. The systems can display the one or more recommendations to the user, and can provide a visual and/or verbal representation to the user that the user can use to make one or more modifications to affect their well-being. The systems can also display the one or more recommendations to third parties (e.g., doctors, therapists, health care workers, fitness coaches, wellness coaches, lifestyle coaches, spiritual guides, religious guides, clinicians, academics (e.g., field experts)), receive feedback from the third parties, and update the one or more recommendations based on the third party input. The third parties can also adjust their treatment/association with the user based on the one or more recommendations. The systems can simulate the user's well-being based at least in part on the user specific parameters and the data from the one or more data sources, the historical user database, or from the reference population. The systems can also monitor the user's well-being over a period of time for identifying trends (e.g., progression, digression, etc.) in the user's well-being, and the one or more recommendations can further be based on the monitoring.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 schematically illustrates a system for affecting a user's well-being:

FIG. 2 is a flow chart process or affecting a user's well-being;

FIG. 3 shows a computer system that is programmed or otherwise configured to implement systems and methods provided herein;

FIG. 4 shows a method of recording, analyzing, and storing user data in accordance with an embodiment of the systems and methods provided herein; and

FIG. 5 shows data acquisition measures and tools recording, analyzing, and storing user data in accordance with an embodiment of the systems and methods provided herein.

### DETAILED DESCRIPTION

While various embodiments of the invention have been shown and described herein, it will be obvious to those skilled in the art that such embodiments are provided by way of example only Numerous variations, changes, and substitutions may occur to those skilled in the art without departing from the invention. It should be understood that various alternatives to the embodiments of the invention described herein may be employed.

### Certain Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a”, “an”, and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter disclosed.

As used herein, the terms “individual(s)”, “subject(s)”, “user(s)”, and “patient(s)” mean any mammal. In some embodiments, the mammal is a human. In some embodiments, the mammal is a non-human.

As used herein, the term “third party” mean any doctor, therapist, health care worker (e.g. a doctor, a registered nurse, a nurse practitioner, a physician's assistant, an orderly or a hospice worker), fitness coach, wellness coach, lifestyle coach, spiritual guide, religious guide, clinician, academic (e.g., a teach or professor), or a researcher (e.g., field expert) associated with a user.

As used herein, the term “user device” means any device used by the user, such as a mobile device, tablet, other personal computing device, or any user connected device (e.g., internet of things device (e.g., associated with the user's environment or daily living (e.g., smart thermostat, smart device, etc.))).

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The terms “effective amount” or “pharmaceutically effective amount” or “therapeutically effective amount” refer to a nontoxic but sufficient amount of the agent to provide the desired biological, therapeutic, and/or prophylactic result. That result might be reduction and/or alleviation of the signs, symptoms, or causes of a disease, or any other desired alteration of a biological system. For example, an “effective amount” for therapeutic uses is the amount of a 5-HT receptor agonist or a pharmaceutically acceptable salt, solvate, metabolite, derivative, or prodrug thereof as disclosed herein per se or a composition comprising a 5-HT receptor agonist or a pharmaceutically acceptable salt, solvate, metabolite, derivative, or prodrug thereof as disclosed herein required to provide a clinically significant decrease in a disease. An appropriate effective amount in any individual case might be determined by one of ordinary skill in the art using routine experimentation.

The term “5-HT receptor agonist agent” refers to a 5-HT receptor agonist as a free base or a derivate or analog thereof. Included in the term are salts, solvates, metabolites, prodrugs, isomers, tautomers, isotopic derivatives, and the like, of a 5-HT receptor agonist. In some embodiments, the derivates, analogs, salts, solvates, metabolites, prodrugs, isomers, tautomers, isotopic derivatives, etc. are pharmaceutically acceptable derivates, analogs, salts, solvates, metabolites, prodrugs, isomers, tautomers, isotopic derivatives of a 5-HT receptor agonist.

The term “pharmaceutically acceptable,” as used herein, refers a material, such as a carrier or diluent, which does not abrogate the biological activity or properties of the one or more drugs, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich: Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting the one or more drugs described herein with a base to provide a “pharmaceutically acceptable base addition salt”. In some embodiments, the one or more drugs described herein is acidic and is reacted with a base. In such situations, an acidic proton of the one or more drugs described herein is replaced by a metal ion, e.g., lithium, sodium, potassium, magnesium, calcium, or an aluminum ion. In some cases, the one or more drugs described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, the one or more drugs described herein form salts with amino acids such as, but not limited to, arginine, lysine, and the like. Acceptable inorganic bases used to form salts with one or more drugs that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the one or more drugs discussed herein are prepared as a sodium salt, calcium salt, potassium salt, magnesium salt, meglumine salt, N-methylglucamine salt or ammonium salt.

In additional or further embodiments, the one or more drugs described herein are metabolized upon administration to an organism in need to produce a metabolite that is then used to produce a desired effect, including a desired therapeutic effect.

A “metabolite” of a drug disclosed herein is a derivative of that drug that is formed when the drug is metabolized. The term “active metabolite” refers to a biologically active derivative of a drug that is formed when the drug is metabolized. The term “metabolized,” as used herein, refers to the sum of the processes (including, but not limited to, hydrolysis reactions and reactions catalyzed by enzymes) by which a particular substance is changed by an organism. Thus, enzymes might produce specific structural alterations to a drug. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Metabolites of the drugs disclosed herein are optionally identified either by administration of drugs to a host and analysis of tissue samples from the host, or by incubation of drugs with hepatic cells in vitro and analysis of the resulting drugs.

The term “treating” and its grammatical equivalents as used herein include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding the fact that the patient might still be afflicted with the underlying disorder. For prophylactic benefit, a method might be performed on, or a composition might be administered to a patient at risk of developing a disease, or to a patient reporting one or more of the physiological symptoms of such conditions, even though a diagnosis of the condition might not have been made.

### 5-HT (or Serotonin) Receptors

The 5-HT (or serotonin) receptors are a group of G protein-coupled receptors (GPCR) and ligand-gated ion channels. 5-HT is short for 5-hydroxy-tryptamine, the chemical name for serotonin.

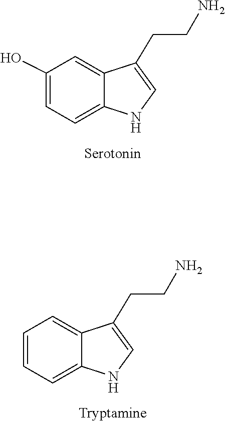 [SMILES: NCCc1c[nH]c2ccc(O)cc12 ; NCCc1c[nH]c2ccccc12]

The serotonin receptors are activated by serotonin, their natural ligand, and mediate both excitatory and inhibitory neurotransmission. They modulate the release of many neurotransmitters, including glutamate, GABA, dopamine, epinephrine/norepinephrine and acetylcholine, as well as many hormones, including oxytocin, prolactin, vasopressin, cortisol, corticotropin and substance P. The serotonin receptors influence various biological and neurological processes such as aggression, anxiety, appetite, cognition, learning, memory, mood, nausea, sleep, and thermoregulation.

The 5-HT receptors are divided into 7 families of G protein-coupled receptors. 5-HT₁, 5-HT₂, 5-HT₃ are the major families; the others, 5-HT₄, 5-HT₅, 5-HT₆ and 5-HT₇, for the most part, work in a similar fashion to either 5-HT: or 5-HT₂ receptors. The 5-HT receptors work with a G protein to modify an ion channel or membrane enzyme.

In certain instances, the 5-HT agonist of a formulation, composition, method, or the like described herein is a S-HT₁ agonist. 5-HT₁ receptors have strong binding affinity for serotonin. Typically, when serotonin binds to a 5-HT₁ receptor, a G-protein is activated, opening an ion channel and allowing potassium ions to exit the neuron. This generally causes the neuron to become more negatively charged, making it more difficult to trigger an action potential, i.e. serotonin binding to 5-HT₁ receptors is an inhibitory effect.

In some preferred embodiments, the 5-HT agonist of a formulation, composition, method, or the like described herein is a 5-HT₂ agonist. In certain instances, the 5-HT₂ agonist has a relatively high affinity for 5-HT₂ receptors (e.g., relative to 5-HT₁ receptors and/or other 5-HT receptors, such as 5-HT₃, 5-HT₄, 5-HT₅, 5-HT₆, 5-HT₇, or all or some combination thereof, such as 2×, 3×, 5×, 10×, 20×, 50×, or the like greater affinity). 5-HT₂ receptors have weaker affinity for serotonin. As such, serotonin prefers to bind 5-HT₁ receptors, typically only binding 5-HT₂ receptors once the 5-HT₁ receptors are at least partially (or wholly) saturated. Serotonin binding of 5-HT₂ receptors typically activates a G-protein closing a potassium channel resulting in potassium ion build up. This generally causes depolarization, making it easier to reach the neuron's excitation threshold. Thus, when serotonin binds to 5-HT₂ receptors, it typically has an excitatory effect.

| Family | Type | Mechanism | Potential |
|---|---|---|---|
| 5-HT₁ | Protein coupled | Decreasing cellular levels of cAMP | Inhibitory |
| 5-HT₂ | Protein coupled | Increasing cellular levels of IP₃ | |
| | | and DAG | |
| 5-HT₃ | Ligand-gated | Depolarizing plasma membrane | Excitatory |
| | Na⁺ and K⁺ | | |
| | cation channel | | |
| 5-HT₄ | Protein coupled | Increasing cellular levels of cAMP | Excitatory |
| 5-HT₅ | Protein coupled | Decreasing cellular levels of cAMP | Inhibitory |
| 5-HT₆ | Protein coupled | Increasing cellular levels of cAMP | Excitatory |
| 5-HT₇ | Protein coupled | Increasing cellular levels of cAMP | Excitatory |

The seven serotonin receptor families include fourteen receptor subtypes, distributed throughout the body as shown in the table below:

| | | Central | Peripheral | | | |
|---|---|---|---|---|---|---|
| 5HT | Blood | nervous | nervous | GI | | Smooth |
| Receptor | vessels | system | system | Tract | Platelets | Muscle |
| 1A | X | X | | | | |
| 1B | X | X | | | | |
| 1D | X | X | | | | |
| 1E | X | X | | | | |
| 1F | | X | | | | |
| 2A | X | X | X | X | X | X |
| 2B | X | X | X | X | X | X |
| 2C | X | X | X | X | X | X |
| 3 | | X | X | X | | |
| 4 | | X | X | X | | |
| 5A | | X | | | | |
| 5B | | | | | | |
| 6 | | X | | | | |
| 7 | X | X | | X | | |

### 5-HT₂ Receptors

In general, 5-HT₂ receptors are characterized by having lower affinity for serotonin (and other indolealkylamines), and are linked to the G_{q}/phospholipase C pathway of signal transduction. In various instances, such receptors mediate a variety of physiological and behavioral functions via three distinct subtypes: 5-HT_{2A}, 5-HT_{2B} and 5-HT_{2C}.

| Receptor | Physiological/behavioral function |
|---|---|
| 5-HT_{2A} | Addiction, Anxiety, Apathy, Appetite, Cognition, Depression, |
| | Imagination, Learning, Memory, Mood, Perception, Sexual |
| | Behavior, Sleep, Thermoregulation, Vasoconstriction |
| 5-HT_{2B} | Anxiety, Appetite, Cardiovascular Function, GI Motility, |
| | Sleep, Vasoconstriction |
| 5-HT_{2C} | Addiction, Anxiety, Appetite, GI Motility, Locomotion, |
| | Mood, Penile Erection, Sexual Behavior, Sleep, |
| | Thermoregulation, Vasoconstriction |

| Receptor | Uses of drugs that act on this receptor |
|---|---|
| 5-HT_{2A} | Antipsychotics , Psychedelics, Noradrenergic and Specific |
| | Serotonergic Antidepressants (NaSSAs), Sleeping aids |
| 5-HT_{2B} | Migraines |
| 5-HT_{2C} | Antidepressant, Orexigenic, Anorectic, Antipsychotic |

| Receptor | Agonists acting on receptor |
|---|---|
| 5-HT_{2A} | Bufotenin, Ergonovine, Lisuride, LSD, Mescaline, |
| | Myristicin, Psilocin, Psilocybin, DMT, DOM, PNU-22394, |
| | TFMPP, 25I-NBOMe, 2C-B, 5-MeO-DMT, BZP |
| 5-HT_{2B} | Fenfluramine, MDMA, Norfenfluramine, Methylphenidate |
| | 6-APB, BW-723C86, PNU-22394, Ro60-0175 |
| 5-HT_{2C} | Aripiprazole, Ergonovine, Lorcaserin, Trazodone |
| | PNU-22394, Ro60-0175, TFMPP, YM-348, A-372, 159, |
| | AL-38022A |

5-HT_{2A} is an important excitatory serotonin receptor subtype. In some instances, physiological processes mediated by the receptor include, by way of non-limiting example:
central nervous system-neuronal excitation, behavioral effects, learning, anxiety, and pro-nociception
smooth muscle contraction (in bronchi and gastrointestinal tract)
vasoconstriction/vasodilation
platelet aggregation
role in memory and learning
anti-inflammatory activity
hormone (oxytocin, prolactin, ACTH, corticosterone, renin) regulation
mood regulation (depressed patients have more 5-HT_{2A} receptors than otherwise normal individuals implying 5-HT_{2A} is involved in the pathogenesis of depression)

In some instances, agonism of 5-HT_{2A} agonism facilitates treatment or management of disorders involving cognitive function and social interaction, or the symptoms thereof, as evidenced by the extensive localization of the 5-HT_{2A} receptor in brain areas that mediate cognitive functions and social interaction. In some instances, disorders in which the 5-HT_{2A} receptor are involved include, but are not limited to schizophrenia, apathy, depression/suicide (e.g., low motivation), anxiety, obsessive compulsive disorders (OCD), bipolar disorders, attention deficit hyperactivity disorder (ADHD), eating disorders such as anorexia nervosa, autism and autism spectrum disorders, Asperger's, neuropsychiatric diseases and disorders, sexual disorders such as erectile dysfunction, neurodegenerative diseases, inflammatory diseases, autoimmune diseases, metabolic diseases such as obesity and diabetes, central nervous system disorders, peripheral nervous system disorders, Alzheimer's disease, snoring, sleep apnea (obstructive sleep apnea, central sleep apnea), insomnia, sleep deprivation, restless legs syndrome, parasomnia, nightmares, night terrors, sleepwalking, hypersomnia (daytime sleepiness), narcolepsy and pain.

Any suitable 5-HT (e.g., 5-HT₂, such as 5-HT_{2A}) agonist is utilized in any composition, formulation, method, therapy, or the like described herein. In some preferred embodiments, the 5-HT agonist of a formulation, composition, method, or the like described herein is a 5-HT_{2A} agonist. In certain instances, the 5-HT_{2A} agonist has a relatively high affinity for 5-HT_{2A} receptors (e.g., relative to 5-HT₁, 5-HT₃, 5-HT₄, 5-HT₅, 5-HT₆, 5-HT₇, 5-HT₂B, 5-HT₂c, or all or some combination thereof, such as 2×, 3×, 5×, 10×, 20×, 50×, or the like greater affinity). In some instances, 5-HT_{2A} agonists increase dopamine levels in the prefrontal cortex. In certain instances, the 5-HT_{2A} agonist provided herein is one of the following classes of S-HT_{2A} agonists: the ergolines, tryptamines and phenethylamines.

### Ergolines

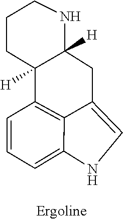 [SMILES: c1cc2c3c(c[nH]c3c1)C[C@H]1NCCC[C@H]21]

In specific embodiments, a 5-HT (e.g., 5-HT_{2A}) receptor agonist utilized herein is an ergoline. In some instances, ergonovine and ergotamine, synthetic derivatives include the oxytocic methergine, the anti-migraine drugs dihydroergotamine and methysergide, hydergine (a mixture of dihydroergotoxine mesylates, INN: ergoline mesylates), and bromocriptine. In certain instances, synthetic ergolines include pergolide and lisuride.

In certain instances, the ergoline is an ergoline derivative, such as a lysergic acid amide or a peptide alkaloid, such as described below. In some instances, the ergoline isa clavine (examples include festuclavine, fumigaclavine A, fumigaclavine B and fumigaclavine C) and other derivatives that do not fall into these categories, such as cabergoline, pergolide, lisuride.

### Lysergic Acid Amides

Exemplary lysergic acid amides include Ergine (LSA, D-lysergic acid amide), Ergonovine (ergobasine), Methergine (ME-277), Methysergide (UML-491), LSD (D-lysergic acid diethylamide), LSH (D-lysergic acid α-hydroxyethylamide). The table below summarizes their structural formula and relationships.

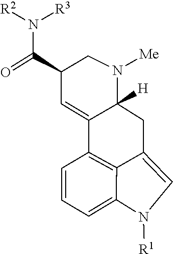

| Name | R¹ | R² | R³ |
|---|---|---|---|
| Ergine | H | H | H |
| Ergonovine | H | CH(CH₃)CH₂OH | H |
| Methergine | H | CH(CH₂CH₃)CH₂OH | H |
| Methysergide | CH₃ | CH(CH₂CH₃)CH₂OH | H |
| LSD | H | CH₂CH₃ | CH₂CH₃ |
| LSH | H | CH(OH)CH₃ | H |

### Peptide Alkaloids

Exemplary peptide alkaloids include, peptide ergot alkaloids (ergopeptines or ergopeptides), which are ergoline derivatives containing a tripeptide structure (attached at the same position as the amide group of the lysergic acid derivatives) comprising proline and two other α-amino acids. Examples include:
Ergotoxines (valine at R²)-Ergocristine, Ergocornine, α-Ergocryptine, β-Ergocryptine
Ergotamines (alanine at R²)-Ergotamine, Ergovaline, α-Ergosine, β-Ergosine.

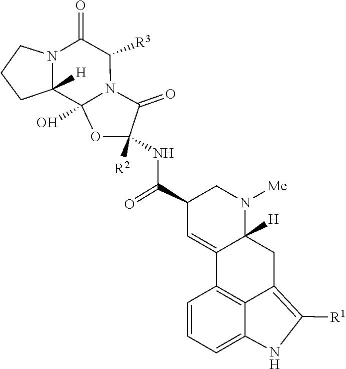

| | | | | R³ Amino |
|---|---|---|---|---|
| Name | R¹ | R² | R³ | acid |
| Ergocristine | | CH(CH₃)₂ | benzyl | Phenylalanine |
| Ergocornine | | CH(CH₃)₂ | CH(CH₃)₂ | Valine |
| α-Ergocryptine | | CH(CH₃)₂ | CH₂CH(CH₃)₂ | Leucine |
| ß-Ergocryptine | | CH(CH₃)₂ | (S)-CH(CH₃)CH₂CH₃ | Isoleucine |
| Ergotamine | | CH₃ | benzyl | Phenylalanine |
| Ergovaline | | CH₃ | CH(CH₃)₂ | Valine |
| α-Ergosine | | CH₃ | CH₂CH(CH₃)₂ | Leucine |
| ß-Ergosine | | CH₃ | (S)-CH(CH₃)CH₂CH₃ | Isoleucine |
| Bromocriptine | Br | CH(CH₃)₂ | CH₂CH(CH₃)₂ | Leucine |

### Tryptamines

Tryptamine (2-(1H-Indol-3-yl) ethanamine) comprises an indole ring, attached to an aminoethylene group; substituted tryptamines are substituted with any suitable group, such as being modified on the indole ring (R¹, R²), the ethylene chain (R³) and/or on the amino group (R⁴, R⁵), as illustrated below, and are collectively referred to herein as tryptamines. Examples of tryptamines include serotonin, melatonin, psilocybin and N,N-Dimethyltryptamine. Additionally, the tryptamine structure may comprise part of a more complex drug, for example: LSD, ibogaine, mitragynine, yohimbine, etc.

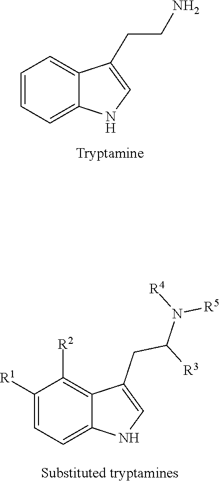

Examples of naturally occurring substituted tryptamines include, by way of non-limiting example:

| Short/Common | | | | | | |
|---|---|---|---|---|---|---|
| Name | Full Name | R¹ | R² | R³ | R⁴ | R⁵ |
| Tryptamine | 3-(2-aminoethyl)indole | H | H | H | H | H |
| | 2-(1H-indol-3- | | | | | |
| | yl)ethanamine | | | | | |
| Bufotenin | 5-hydroxy-N,N- | OH | H | H | CH₃ | CH₃ |
| | dimethyltryptamine | | | | | |
| Nω-Methylserotonin | 5-hydroxy-N- | OH | H | H | CH₃ | H |
| (norbufotenin) | methyltryptamine | | | | | |
| Serotonin | 5-hydroxytryptamine | OH | H | H | H | H |
| NMT | N-methyltryptamine | H | H | H | H | CH₃ |
| 5-MeO-NMT | 5-methoxy-N- | OCH₃ | H | H | CH₃ | H |
| | methyltryptamine | | | | | |
| DMT | N,N-dimethyltryptamine | H | H | H | CH₃ | CH₃ |
| 5-Bromo-DMT | 5-bromo-N,N- | Br | H | H | CH₃ | CH₃ |
| | dimethyltryptamine | | | | | |
| 5-MeO-DMT | 5-methoxy-N,N- | OCH₃ | H | H | CH₃ | CH₃ |
| | dimethyltryptamine | | | | | |
| Melatonin | 5-methoxy-N- | OCH₃ | H | H | C(O)CH₃ | H |
| | acetyltryptamine | | | | | |
| N-Acetylserotonin | 5-hydroxy-N- | OH | H | H | C(O)CH₃ | H |
| | acetyltryptamine | | | | | |
| Norbaeocystin | 4-phosphoryloxy- | H | OPO₃H₂ | H | H | H |
| | tryptamine | | | | | |
| Baeocystin | 4-phosphoryloxy-N- | H | OPO₃H₂ | H | CH₃ | H |
| | methyl-tryptamine | | | | | |
| Psilocybin | 4-phosphoryloxy-N,N- | H | PO₄ | H | CH₃ | CH₃ |
| | dimethyltryptamine | | | | | |
| Psilocin | 4-hydroxy-N,N- | H | OH | H | CH₃ | CH₃ |
| | dimethyltryptamine | | | | | |
| Tryptophan | α-carboxyltryptamine | H | H | COOH | H | H |

Examples of synthetic substituted tryptamines include, by way of non-limiting example:

| Short | | | | | | |
|---|---|---|---|---|---|---|
| Name | Name | R¹ | R² | R³ | R⁴ | R⁵ |
| αET | α-ethyltryptamine | H | H | CH₂CH₃ | H | H |
| αMT | α-methyltryptamine | H | H | CH₃ | H | H |
| DALT | N,N-diallyltryptamine | H | H | H | H₂C═CH—CH₂ | H₂C═CH—CH₂ |
| DET | N,N- | H | H | H | CH₂CH₃ | CH₂CH₃ |
| | diethyltryptamine | | | | | |
| DiPT | N,N- | H | H | H | CH(CH₃)₂ | CH(CH₃)₂ |
| | diisopropyltryptamine | | | | | |
| DPT | N,N- | H | H | H | CH₂CH₂CH₃ | CH₂CH₂CH₃ |
| | dipropyltryptamine | | | | | |
| 5-MeO- | 5-methoxy-α- | OCH₃ | H | CH₃ | H | H |
| αMT | methyltryptamine | | | | | |
| 5-MeO- | 5-methoxy-N,N- | OCH₃ | H | H | H₂C═CH—CH₂ | H₂C═CH—CH₂ |
| DALT | diallyltryptamine | | | | | |
| 5-MeO- | 5-methoxy-N-Methyl- | OCH₃ | H | H | H₂C═CH—CH₂ | CH₃ |
| MALT | N-allyltryptamine | | | | | |
| 4-HO- | 4-hydroxy-N,N- | H | OH | H | CH₂CH₃ | CH₂CH₃ |
| DET | diethyltryptamine | | | | | |
| 4-AcO- | 4-acetoxy-N,N- | H | OCOCH₃ | H | CH₃ | CH₃ |
| DMT | dimethyltryptamine | | | | | |
| 4-HO- | 4-hydroxy-N-methyl- | H | OH | H | CH₃ | CH₂CH₃ |
| MET | N-ethyltryptamine | | | | | |
| 4-HO- | 4-hydroxy-N,N- | H | OH | H | CH(CH₃)₂ | CH(CH₃)₂ |
| DIPT | diisopropyltryptamine | | | | | |
| 5-MeO- | 5-methoxy-N,N- | OCH₃ | H | H | CH(CH₃)₂ | CH(CH₃)₂ |
| DIPT | diisopropyltryptamine | | | | | |
| 5-MeO- | 5-methoxy- | OCH₃ | H | H | CH₃ | CH(CH₃)₂ |
| MiPT | N,N-methyliso- | | | | | |
| | propyltryptamine | | | | | |
| 4-HO- | 4-hydroxy-N- | H | OH | H | CH(CH₃)₂ | CH₃ |
| MiPT | isopropyl-N- | | | | | |
| | methyltryptamine | | | | | |
| Suma- | 5-(methylamino | CH₂SO₂NHCH₃ | H | H | CH₃ | CH₃ |
| triptan | sulfonylmethylene)- | | | | | |
| | N,N- | | | | | |
| | dimethyltryptamine | | | | | |
| Zolmi- | 5-(4-(S)-1,3- | CHNHC(O)OCH₂ | H | H | CH₃ | CH₃ |
| triptan | oxazolidin- | | | | | |
| | 2-one)-N,N- | | | | | |
| | dimethyltryptamine | | | | | |

### Phenethylamines

Phenethylamine comprises a phenyl ring attached to an aminoethylene group; substituted phenethylamines are optionally substituted in any suitable manner, such as they are optionally modified by substitution on the phenyl ring (R¹, R², R³, R⁴ and/or R⁵), the ethylene chain (R⁶ and/or R⁷) and/or on the amino group (R⁸, and/or R⁹), such as illustrated below.

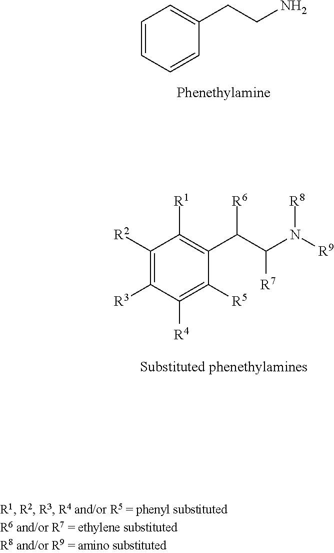

Examples of phenethylamines include, but are not limited those presented in the table below:

| | | Substitution | | | Biological |
|---|---|---|---|---|---|
| Short Name | Full Name | Amino | Ethylene | Phenyl | activity |
| Amphetamine | α- | | X | | Stimulant |
| | methylphenethylamine | | | | |
| β-Methyl | β- | | X | | Stimulant |
| phenethylamine | methylphenethylamine | | | | |
| Mephedrone | 4-methylmethcathinone | X | X | X | Stimulant |
| Ethcathinone | N-ethylcathinone | X | X | | Stimulant |
| Ephedrine/ | N-methyl-β- | X | X | | Stimulant; |
| Pseudoephedrine | hydroxyamphetamine | | | | decongestant |
| Methamphetamine | N-methylamphetamine | X | X | | Stimulant; |
| | | | | | neurotoxin |
| Phentermine | α-methylamphetamine | | X | | Stimulant, |
| | | | | | anorectic |
| Ortetamine | 2-methylamphetamine | | X | X | Stimulant, |
| | | | | | anorectic |
| Amfepramone | N-diethyl-β- | X | X | | Anorectic |
| (diethylpropion) | ketoamphetamine | | | | |
| Phenylephrine | β,3-dihydroxy-N- | X | X | X | Decongestant |
| | methylphenethylamine | | | | |
| Methylphenidate | N,α-butylene-β-methoxy | X | X | | Stimulant; NDRI |
| | carbonylphenethylamine | | | | |
| Dopamine | 3,4- | | | X | Catecholamine |
| | dihydroxyphenethylamine | | | | neurotransmitter |
| 6- | 2,4,5-trihydroxy | | | X | Neurotoxic |
| Hydroxydopamine | phenethylamine | | | | agent |
| Epinephrine | β-3,4-trihydroxy-N- | X | X | X | Catecholamine |
| (Adrenaline) | methylphenethylamine | | | | neurotransmitter |
| Norepinephrine | β-3,4-trihydroxy | | X | X | Catecholamine |
| (Noradrenaline) | phenethylamine | | | | neurotransmitter |
| para-Octopamine | β-4-dihydroxy | | X | X | Trace aminergic |
| | phenethylamine | | | | α-adrenoceptor |
| | | | | | agonist |
| Salbutamol | β-4-dihydroxy-3- | X | X | X | Short-action β2- |
| | hydroxymethyl-N-tert- | | | | adrenergic |
| | butylphenethylamine | | | | agonist |
| N-Methyl | N- | X | | | Amphetamine |
| phenethylamine | methylphenethylamine | | | | isomer |
| Cathine | d-β- | | X | | Releasing agent |
| | hydroxyamphetamine | | | | |
| Cathinone | β-ketoamphetamine | | X | | Releasing agent |
| Methcathinone | N-methylcathinone | X | X | | Releasing agent |
| Bupropion | 3-chloro-N-tert-butyl-β- | X | X | X | NDRI |
| | ketoamphetamine | | | | |
| Norfenfluramine | 3-trifluoromethyl- | | X | X | SSRA |
| | amphetamine | | | | |
| Fenfluramine | 3-trifluoromethyl-N- | X | X | X | SSRA |
| | ethylamphetamine | | | | |
| Mescaline | 3,4,5-trimethoxy | | | X | Psychedelic |
| | phenethylamine | | | | |
| Proscaline | 2-(3,5-dimethoxy-4- | | | X | Psychedelic |
| | propoxyphenyl)ethanamine | | | | |
| Metaescaline | 2-(3-ethoxy-4,5- | | | X | Psychedelic |
| | dimethoxyphenyl)ethanamine | | | | |
| Allylescaline | 4-Allyloxy-3,5- | | | X | Psychedelic |
| | dimethyloxy | | | | |
| | phenylethylamine | | | | |
| Methallylescaline | 4-Methallyloxy-3,5- | | | X | Psychedelic |
| | dimethoxyphenethylamine | | | | |
| Asymbescaline | 3,4-Diethoxy-5- | | | X | Psychedelic |
| | methoxyphenethylamine | | | | |

The present disclosure provides systems and methods for affecting a user's well-being. Provided in certain embodiments herein is a method for affecting a user's well-being. In some embodiments, the method comprises receiving data from one or more data sources. In some embodiments, the method further comprises processing the data received from the one or more data sources to generate or extract one or more user specific parameters from the received data. In some embodiments, the method further comprises initializing one or more models of well-being incorporating data from at least a reference population or a historical user database, and generating an output indicative of a state of the user's well-being based at least in part on the user specific parameters and the data from at least the reference population or the historical user database using the one or more models. In some embodiment, the method comprises: (a) receiving data from one or more data sources; (b) processing the data received from the one or more data sources to generate or extract one or more user specific parameters from the received data; and (c) initializing one or more models of well-being incorporating data from at least a reference population or a historical user database, and generating an output indicative of a state of the user's well-being based at least in part on the user specific parameters and the data from at least the reference population or the historical user database using the one or more models.

Provided in some embodiments herein is a method for affecting a user's well-being comprising: (a) initializing one or more models of well-being incorporating data from at least a reference population or a historical user database, (b) generating user specific parameters relating to the user's well-being, and (c) simulating the user's well-being based at least in part on the user specific parameters and the reference population data or the data in the historical user database. In some embodiments, the method may further comprise (d) updating the one or more models based on newly received data from the user in (b) and newly received data from the reference population or the historical user database. In some embodiments, the method may further comprise (e) generating one or more recommendations to the user to affect (e.g., improve) the user's well-being.

Provided in some embodiments herein is a computer-implemented method for affecting a user's well-being, comprising (a) receiving data from one or more data sources; (b) processing the data received from the one or more data sources to generate or extract one or more user specific parameters from the received data; and (c) initializing one or more models of well-being incorporating data from at least a reference population or a historical user database, and generating an output indicative of a state of the user's well-being based at least in part on the user specific parameters and the data from at least the reference population or the historical user database using the one or more models.

Provided in some embodiments herein are one or more non-transitory computer storage media storing instructions that are operable, when executed by one or more computers, to cause the one or more computers to perform operations comprising: (a) receiving data from one or more data sources; (b) processing the data received from the one or more data sources to generate or extract one or more user specific parameters from the received data; and (c) initializing one or more models of well-being incorporating data from at least a reference population or a historical user database, and generating an output indicative of a state of the user's well-being based at least in part on the user specific parameters and the data from at least the reference population or the historical user database using the one or more models.

Provided in some embodiments herein is a system comprising one or more computers and one or more storage devices storing instructions that are operable, when executed by the one or more computers, to cause the one or more computers to perform operations comprising: (a) receiving data from one or more data sources; (b) processing the data received from the one or more data sources to generate or extract one or more user specific parameters from the received data; and (c) initializing one or more models of well-being incorporating data from at least a reference population or a historical user database, and generating an output indicative of a state of the user's well-being based at least in part on the user specific parameters and the data from at least the reference population or the historical user database using the one or more models.

In any of the embodiments provided herein, affecting the user's well-being comprises improving the user's well-being. In any of the embodiments provided herein, affecting the user's well-being comprises improving the user's health outcomes. In any of the embodiments provided herein, improving the user's health outcomes comprises improving a user's state in regards to one or more of the following emotions: alertness, excitement, elation, happiness, contentment, relaxation, calmness, sleepiness, fatigue, boredom, depression, sadness, frustration, stress, nervousness, tenseness, or a combination thereof. In any of the embodiments provided herein, improving the user's health outcomes can comprise improving a user's ability to cope with or overcome a disorder or condition, for example, a neurological condition the user is suffering from. In any of the embodiments provided herein, the neurological condition is a neurological disorder. In some embodiments, the neurological condition is a neurocognitive disorder. In some embodiments, the symptoms of the neurological condition are physical, behavioral, emotional, mental or a combination thereof. In some embodiments, the neurological condition is an addictive disorder. In some embodiments, the addictive disorder is alcohol abuse, substance abuse, smoking, or obesity. In some embodiments, the neurological condition is an eating disorder or an auditory disorder. In some embodiments, the neurological condition is pain (e.g. chronic pain). In some embodiments, the neurological condition is depression, bipolar disorder, anxiety, social anxiety, post-traumatic stress disorder (PTSD), panic disorder, phobia, schizophrenia, psychopathy, or antisocial personality disorder. In some embodiments, the neurological condition is an impulsive disorder. In some embodiments, the impulsive disorder is attention deficit hyperactivity disorder (ADHD), attention deficit disorder (ADD), Tourette's syndrome or autism. In some embodiments, the neurological condition is a compulsive disorder. In some embodiments, the compulsive disorder is obsessive compulsive disorder (OCD), gambling, or aberrant sexual behavior. In some embodiments, the neurological condition is a personality disorder. In some embodiments, the personality disorder is conduct disorder, antisocial personality, or aggressive behavior.

In any of the embodiments provided herein, the disorders or conditions are neurological disorders or conditions. In some embodiments, the disorders or conditions are neurocognitive disorders or conditions. In some embodiments, the disorders or conditions are neurodegenerative disorders or conditions. In some embodiments, the symptoms of the neurological condition are physical, behavioral, emotional, mental, or a combination thereof.

In any of the embodiments provided herein, the methods may improve a user's ability to cope with or overcome disorders, conditions or symptoms, which may include but not be limited to addiction disorders, such as but not limited to alcohol abuse, substance abuse, smoking, or obesity. In any of the embodiments provided herein, the methods may improve a user's ability to cope with or overcome disorders, conditions or symptoms including but not limited to eating disorders and auditory disorders. In any of the embodiments provided herein, the methods may improve a user's ability to cope with or overcome disorders, conditions or symptoms including but not limited to pain, such as but not limited to chronic pain. In any of the embodiments provided herein, the methods may improve a user's ability to cope with or overcome disorders, conditions or symptoms including but not limited to depression, bipolar disorder, post-traumatic stress disorder (PTSD), panic disorder, phobia, schizophrenia, psychopathy, or antisocial personality disorder. In any of the embodiments provided herein, the methods may improve a user's ability to cope with or overcome disorders, conditions or symptoms including but not limited to impulse disorders, such as but not limited to attention deficit hyperactivity disorder (ADHD), Tourette's syndrome or autism. In any of the embodiments provided herein, the methods may improve a user's ability to cope with or overcome disorders, conditions or symptoms including but not limited to compulsive disorder, such as but not limited to obsessive compulsive disorder (OCD), gambling, or aberrant sexual behavior. In any of the embodiments provided herein, the methods may improve a user's ability to cope with or overcome disorders, conditions or symptoms including but not limited to personality disorders, such as but not limited to conduct disorder, antisocial personality, or aggressive behavior.

In any of the embodiments provided herein, the reference population data, the historical user data, or a combination thereof, are gathered over a time period. In any of the embodiments provided herein, the time period comprises at least 1 day (e.g., 2, 3, 4, 5, 6, or 7 days). In any of the embodiments provided herein, the time period comprises at least 1 week (e.g., 2, 3, or 4 weeks). In any of the embodiments provided herein, the time period comprises at least 1 month (e.g., 2, 3, 4, 5, 6, 7, 8, 9, 10, 11, or 12 months). In any of the embodiments provided herein, the time period comprises at least 1 year (e.g., 2, 3, 4, 5, 6, 7, 8, 9, 10, 11, or 12 years). In any of the embodiments provided herein, the reference population data, the historical user database, or a combination thereof, are received from a third party library. In any of the embodiments provided herein, the reference population data is gathered by: (i) receiving data from one or more data sources associated with a reference population, and (ii) processing the data received from the one or more data sources. In any of the embodiments provided herein, the historical user database is gathered over the time period according to the method of claim 1. For example, the historical user database can be gathered by receiving data from one or more data sources. Gathering the historical user database may also comprise processing the data received from the one or more data sources to generate or extract one or more user specific parameters from the received data. Gathering the historical user database may also comprise initializing one or more models of well-being incorporating data from at least a reference population or a historical user database, and generating an output indicative of a state of the user's well-being based at least in part on the user specific parameters and the data from at least the reference population or the historical user database using the one or more models.

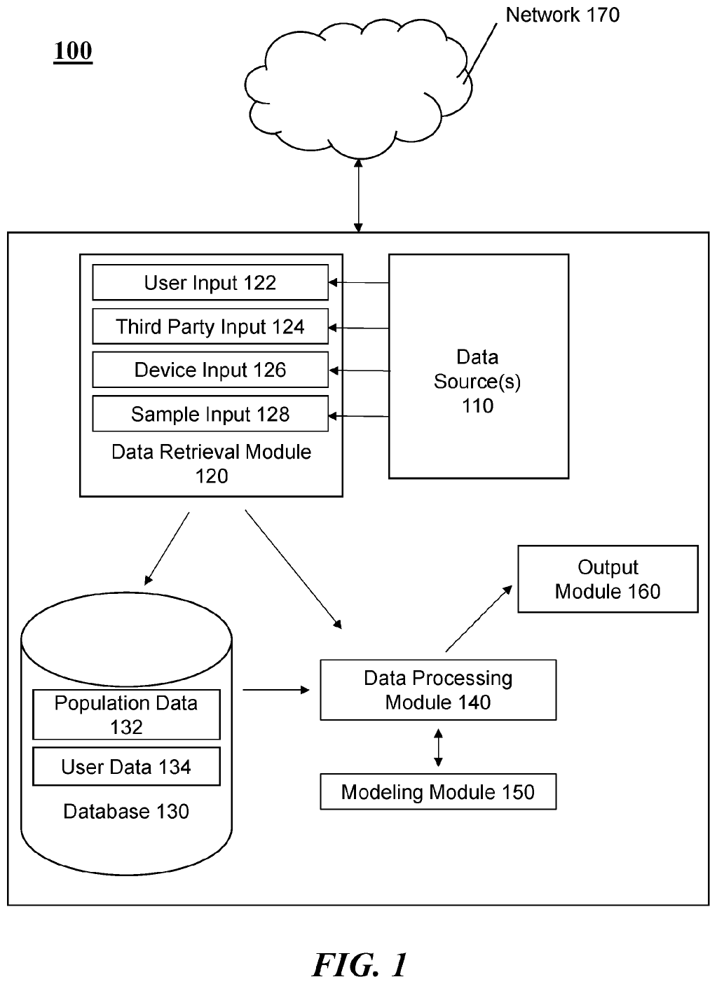

FIG. 1 schematically illustrates a system 100 for affecting a user's well-being, in accordance with some embodiments of the present disclosure. The system 100 may have a data source(s) 110, a data retrieval module 120, a database 130, a data processing module 140, a modeling module 150, an output module 160, and a network 170. The system can perform the method of any one of the embodiments described herein.

The system 100 can receive data from one or more data sources 110. The system 100 can receive the data using the data retrieval module 120. The data retrieval module 120 can receive, as a non-limiting example, user input 122, third party input 124, device input 126, and/or sample input 128.

The system 100 can store the data from the one or more data sources in the database 130. The database may include, for example, raw data collected and received from the one or more data sources. The system 100 can store the user input 122, third party input 124, device input 126, and/or sample input 128 in the user data 134 portion of the database. The compiled user data 134 in the database 130 over time can serve as a historical user database. The user data 134 can also be acquired from a third party library. The historical user database can be used in embodiments described herein for initializing models and for data comparison for generating outputs associated with the user's well-being. The database 130 can also store population data 132. The population data 132 can be data from a reference population. The reference population data can be acquired using the data retrieval module for a plurality of users of the system over time. The reference population can include data from people in a similar situation as the user(s). For example, the reference population data can include data from people suffering with one or more mental, physical, emotional, or social disorders. The database 130

The data processing module 140 can comprise one or more data processing algorithms described herein for processing the data received from the one or more data sources 110. The data processing algorithms can comprise, as a non-limiting example, feature extraction algorithms, machine learning algorithms, artificial intelligence algorithms, Bayesian algorithms (e.g., Bayesian assimilation, Bayesian estimation), and/or statistical analysis algorithms. The data processing module can process data in real-time, near real-time, or in a dynamic nature. The modeling module 150 can be used to initiate one or more models as described herein. For example, the one or more models can include pre-programmed models, artificial intelligence models, machine learning models, or a combination thereof. The output module 160 can be used for generating an output (e.g., one or more recommendations) to a user of the system that is associated with affecting or improving a user's well-being. The output module 160 can output the one or more recommendations to a user using a graphical user interface on a user's device. The recommendation can be presented as a visual representation or a textual representation, or a combination of the two. The network 170 can ensure that the components of system 100 are in communication with one another. The components of system 100 can be implemented on a local hard drive. The components of system 100 can be implemented on the cloud. The components of system 100 can be implemented on a combination of local hard drives and the cloud. The system 100 can be operatively coupled to the network 170 with the aid of a communication interface. The network 170 can be the Internet, an internet and/or extranet, or an intranet and/or extranet that is in communication with the Internet. The network 170 in some cases is a telecommunication and/or data network. The network 170 can include one or more computer servers, which can enable distributed computing, such as cloud computing. The network 170, in some cases with the aid of the system 100, can implement a peer-to-peer network, which may enable devices coupled to the system 100 to behave as a client or a server.

The system 100 can communicate with one or more remote computer systems through the network 170. For instance, the system 100 can communicate with a remote computer system of a user (e.g., a user device, as may be described herein), a third party (e.g., a third party device (e.g., a third party phone or tool (e.g., doctor portal) for use in assessing the user's well-being)), a third party laboratory (e.g., clinical, hospital, bio bank, or outpatient laboratory). Examples of remote computer systems include personal computers (e.g., portable PC), slate or tablet PC's (e.g., Apple® iPad, Samsung Galaxy Tab), telephones, Smart phones (e.g., Apple® iphone, Android-enabled device, Blackberry®), or personal digital assistants. The user can access the system 100 via the network 170.

The remote computer system may include a display. The remote computer system may include one or more wearable devices, one or more implantable devices, or a combination thereof, as described herein. The display may be a screen. The display may or may not be a touchscreen. The display may be a light-emitting diode (LED) screen, OLED screen, liquid crystal display (LCD) screen, plasma screen, or any other type of screen. The display may be configured to show a user interface (UI) or a graphical user interface (GUI) rendered through an application (e.g., via an application programming interface (API) executed on the user device). The GUI may show graphical elements that permit a user to monitor collected sensor data, generated scores, view a notification or report regarding the user's well-being state, view queries prompted by health care provider regarding determined well-being state, and/or view one or more recommendations to affect or improve the user's well-being.

The system 100 of FIG. 1 and its components can be implemented on one or more computing devices. The computing devices can be servers, desktop or laptop computers, electronic tablets, mobile devices, or the like. The computing devices can be located in one or more locations. The computing devices can have general-purpose processors, graphics processing units (GPU), application-specific integrated circuits (ASIC), field-programmable gate-arrays (FPGA), or the like. The computing devices can additionally have memory, e.g., dynamic or static random-access memory, read-only memory, flash memory, hard drives, or the like. The memory can be configured to store instructions that, upon execution, cause the computing devices to implement the functionality of the subsystems. The computing devices can additionally have network communication devices. The network communication devices can enable the computing devices to communicate with each other and with any number of user devices, over a network. The network can be a wired or wireless network. For example, the network can be a fiber optic network, Ethernet® network, a satellite network, a cellular network, a Wi-Fi® network, a Bluetooth® network, or the like. In other implementations, the computing devices can be several distributed computing devices that are accessible through the Internet. Such computing devices may be considered cloud computing devices.

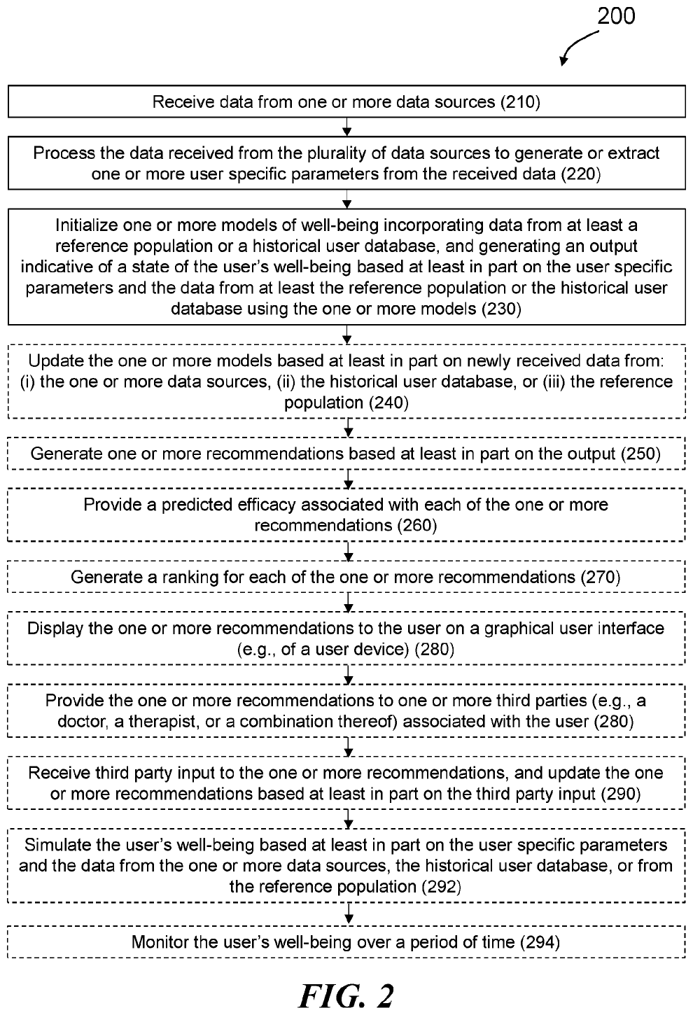

FIG. 2 is a flowchart of a process 200 for affecting a user's well-being. The process 200 can be performed by a system of one or more appropriately programmed computers in one or more locations. For example, the process can be performed by the system 100 of FIG. 1.

The system can receive data from one or more data sources (210). In any of the embodiments provided herein, the one or more data sources comprises a plurality of data sources. In any of the embodiments provided herein, the one or more data sources each being independently selected from the group consisting of user input, third party input, device input, and sample input. Any of the one or more data sources can provide physiological, neurological, psychological, metabolic, or biological data about a user useful for identifying a state of the user's well-being and for recommending one or more recommendations to the user for affecting or improving the user's well-being.

In any of the embodiments provided herein, the user input comprises data received from one or more user devices (e.g., a mobile device, tablet, or other personal computing device, or any or any user connected device (e.g., internet of things device (e.g., associated with the user's environment or daily living (e.g., smart thermostat, smart device, etc.))). For example, the user device may be the user's cell phone, laptop or desktop computer, or a portable tablet. As an example, the connected devices can be any device connected to and receiving data about the user's well-being, health, environment (e.g., work, home, school, or living environment). For example, the connected device may be a smart thermostat in the user's house, and the system can acquire temperature, humidity, and other information from the smart thermostat in use for determining a state of the user's well-being. For example, a higher temperature in the user's living environment can indicate a higher likelihood of anxiety, and a lower temperature in the user's living environment can indicate a user is more likely to be calm.

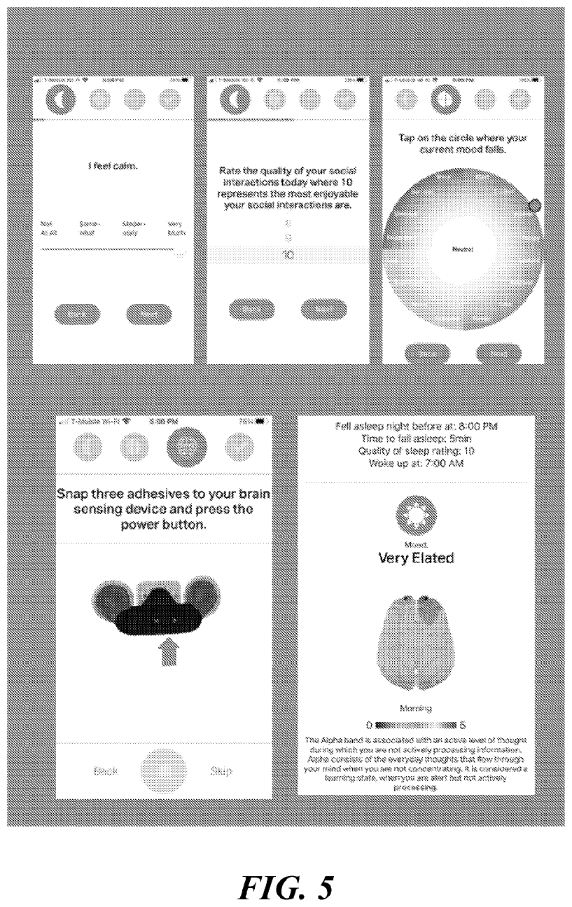

In any of the embodiments provided herein, receiving the data comprises administering one or more surveys or questionnaires to the user. In any of the embodiments provided herein, the user input comprises user responses to the one or more surveys or questionnaires. For example, the questionnaires can be designed to acquire data about the user feeling alert, excited, elated, happy, content, relaxed, calm, sleepy, fatigued, bored, depressed, sad, frustrated, stressed, nervous, and/or tense. In any of the embodiments provided herein, the user input comprises quantitative, qualitative, or a combination thereof, input. Qualitative input can include a user's written response to the questionnaire or prompts associated with the questionnaires. Quantitative input can include a score on a scale, for example 0-10, provided by the user. FIG. 5 provides examples of questionnaires and the user's available options for providing answers. For example, the questionnaires can be formal questionnaires designed for gathering specific data about the user, such as a level of depression, anxiety, or stress in the user. As an additional example, the questionnaires can be questions enquiring aspects about the user's daily life. For example, the questionnaire prompts can include: I feel calm; I am tense; I am upset; I am relaxed; I am content; and/or I am worried. User response options to the prompts can include: Not at all; Somewhat; Moderately; or Very Much. As an additional example, the question may ask the user to “Rate the quality of your social interactions today where 10 represents the most enjoyable your social interactions are,” and the user can provide an answer on a scale of 0-10. As an additional example, the question may ask the user to “Rate your level of fatigue where 10 represents the most fatigued you get,” and the user can provide an answer on a scale of 0-10. As an additional example, the question may ask the user to “Rate your ability to maintain your focus and attention today where 10 represents the most attentive and focused you can be,” and the user can provide an answer on a scale of 0-10. As an additional example, the question may ask the user to “Rate your quality of sleep last night where 10 is your best quality of sleep,” and the user can provide an answer on a scale of 0-10. As an additional example, the questionnaires may ask the user if they have taken any psychoactive substances, prescribed medication, caffeine, nicotine, marijuana, other drugs, alcohol, dietary supplements, or the like, and the user's answer can be a yes or no. As an additional example, the questionnaire may ask a user to identify where their current mood falls, and the user's response can be an indication via the user device on a graphical user interface where the user's mood falls on a scale (e.g., neural, alert, excited, elated, happy, content, relaxed, calm, sleepy, fatigued, bored, depressed, sad, frustrated, stressed, nervous, and/or tense). As an additional example, the questionnaires can ask the user what time the user went to sleep the night before, what time they woke up that morning, and the user can provide an answer of a specific time. As an additional example, the questionnaires can ask the user how long it took for the user to fall asleep the night before, and the user can provide a number answer related to how many minutes, or hours, it took the user to fall asleep.

In any of the embodiments provided herein, the one or more surveys or questionnaires are administered at least once (e.g., twice, three times, four times, five times, six times, seven times, eight times, nine times, or ten times) daily to the user. In any of the embodiments provided herein, the one or more surveys or questionnaires are administered at least once (e.g., twice, three times, four times, five times, six times, seven times, eight times, nine times, or ten times) a week to the user. In any of the embodiments provided herein, the one or more surveys or questionnaires are administered at least once (e.g., twice, three times, four times, five times, six times, seven times, eight times, nine times, or ten times) a month to the user. In some embodiments, questionnaires are administered once, twice, three times, four times, five times, six times, seven times, eight times, nine times, ten times, eleven times, twelve times, thirteen times, or fourteen times or more weekly for at least 1, 2, 3, 4, 5, 6, 7, 8, 9, 10, 11, or 12 or more weeks, or for 4, 5, 6, 7, 8, 9, 10, 11, 12 or more months, or for 1, 2, 3, or 4 or more years.

In any of the embodiments provided herein, the one or more surveys or questionnaires comprises surveys acquiring emotional data of the user. In any of the embodiments provided herein, the emotional data comprises data relating to depression, anxiety, stress, coping, mood, sleep, attention, quality of life, phobias, demoralization, rumination, social interaction (e.g., a quality or a quantity thereof), or a combination thereof, of the individual. In any of the embodiments provided herein, the emotional data comprises data associated with one or more of the following emotions: alertness, excitement, elation, happiness, contentment, relaxation, calmness, sleepiness, fatigue, boredom, depression, sadness, frustration, stress, nervousness, tenseness, or a combination thereof. For example, the questionnaires can ask the user to “Rate their current level, or level in the last day, week, month, or year, of depression, anxiety, stress, coping, mood, sleep, attention, and/or quality of life, and the user can provide an answer on a scale from 0-10. In any of the embodiment provided herein, the scale may be 0-10, 0-5, 0-100, or the like.

In any of the embodiments provided herein, the one or more surveys or questionnaires comprises surveys acquiring physical data of the user. In any of the embodiments provided herein, the physical data comprises data relating to the user's physiological state (e.g., weight, height, diet, arousal, sleep, dreams), physical exercise (e.g., amount of physical exercise), substance consumption, or a combination thereof. For example, the questionnaires can ask for the user's weight, height, and other physical characteristics in order to track the user's physical characteristics and determine trends over time. As an additional example, the questionnaires can ask a user to identify a number of hours the user has exercised in the last day or week. The questionnaires can also ask the user to identify the quality of the physical exercise, what parts of the body the user exercised, and how they felt after exercising.

In any of the embodiments provided herein, the one or more surveys or questionnaires comprises surveys acquiring data of one or more stressors of the user. In any of the embodiments provided herein, the one or more stressors comprises data relating to the user's daily interactions (e.g., commute, number of meetings at work, exams in school, etc.). For example, a user may identify they had a higher number of meeting at work than average, and this information could be useful in identifying an increased state of anxiety or stress in the user. The stressors can be any events or interactions the user came across in the last day or week in which the user's state of stress increased. The questionnaire can ask the user to identify incidents to be considered as stressors, and the user can also provide a number on a scale from 0-10 how stressed he user felt after the incident.

In any of the embodiments provided herein, the one or more stressors comprises data relating to long term stress comprising stressors associated with family, social life, financial status, health, accidents, response to news, or a combination thereof. For example, an illness of a family member, a dissipating social life (e.g., which may be identified as a trend based on user answers to questionnaires enquiring about the user's social interactions), financial struggles, personal health problems, and news of major world events could cause long term stress in the user. As an additional example, the birth of a baby, a strong social life (e.g., which may be identified as a trend based on user answers to questionnaires enquiring about the user's social interactions), financial successes, personal health improvements, and news of major world events could lead to sustained feelings of happiness and elation in the user.

In any of the embodiments provided herein, the one or more surveys or questionnaires comprises a Beck Depression Inventory (BDI), a Generalized Anxiety Disorder (GAD-7), a Depression Anxiety Stress Scale (DASS), a Brief-COPE, a Positive and Negative Affect Schedule (PANAS), a State Trait Anxiety Inventory (STAI), a modified version of a Russel Mood Circumplex, a modified version of a NIH Sleep Diary, a 36 Item Short Form Health Survey (SF-36), a 5D Altered state of Consciousness Scale (5d-ASC), a daily sleep diary questionnaire, a Hamilton Rating Scale for Depression, a Hamilton Anxiety Rating Scale, mood surveys, self-reporting surveys (e.g., with questions about attention, social interactions, fatigue), sleep diaries or a combination thereof.

In any of the embodiments provided herein, the user input comprises the user's unprompted input related to the user's well-being (e.g., input relating to the user's social interactions, emotional state, physical state, one or more stressors, input to a user diary, or a combination thereof). In any of the embodiments provided herein, the unprompted input comprises quantitative, qualitative, or a combination thereof, input. In any of the embodiments provided herein, the user's unprompted input comprises a self-examination of the user's well-being. For example, the user's unprompted input can be entered and/or stored into a user diary that the user writes to each day or week. If a user is not administered a questionnaire about the user's stress levels that day, then the user can self-report if the user is feeling particularly stressed or particularly calm. As an additional example, the user can provide unprompted input using a visual representation of a human body and a graphical user interface of a user device. The user can select portions of the body where they feel pain, they do not feel good, or they feel they need improvement. These selected body parts can be selected in a color indicating a need for help, for example, in red. The user can also identify body parts where they feel health, strong, or in no need of improvement. For example, the user can highlight the head and heart in red if the user is feeling depressed and the user is experiencing higher heart rate than normal. As an additional example, the user can highlight the head and heart green if the user is feeling happy or elated and the user was able to get physical exercise in that day. In any of the embodiments provided herein, the unprompted input can comprise input related to one or more of the following emotions: alertness, excitement, elation, happiness, contentment, relaxation, calmness, sleepiness, fatigue, boredom, depression, sadness, frustration, stress, nervousness, tenseness, or a combination thereof.

In any of the embodiments provided herein, the answers to the questionnaires, and/or the user provided unprompted input, can be useful for storing data from the user that can provide information on a state of the user's well-being. The data can be analyzed as is it being received for real-time, near real-time, or dynamically determining a state of the user's well-being and generating one or more recommendations of how to affect or improve the user's well-being. The data can also be stored over time to compile a historical user database for each and every user. The historical user database can be used to identify trends in the user's well-being over time. The trends can be used to help determine a state of the user at any given time and to generate one or more personalized recommendations for affecting or improving the user's well-being.

In any of the embodiments provided herein, the third party input comprises data from the user's doctor(s), family, friends, co-workers, therapists, counselors, teachers, professors, spiritual guides, religious guides, fitness coach, wellness coach, lifestyle coach, healthcare workers, or a combination thereof. For example, the system can receive data from the user's therapist regarding the user's mental state, including the user's current level of depression, anxiety, stress, coping, mood, sleep, attention, and quality of life. The system can receive data from the user's friends or family regarding the user's recent life activities and if the user has shown any signs of increased or decreased well-being. In any of the embodiments provided herein, the third party input comprises doctor input (e.g., observations (e.g., after a doctor-user visit)) on the user's well-being. For example, a user may visit their doctor, the doctor may determine that the user is improving or declining in a certain aspect of the user's well-being and/or health, and the doctor can provide that information to the system for processing. In any of the embodiments provided herein, the third party input comprises input (e.g., observations) from the user's family, friends, co-workers, therapists, counselors teachers, professors, spiritual guides, religious guides, religious guides, fitness coach, wellness coach, lifestyle coach, healthcare workers, or a combination thereof. As an example, the system can receive information from any of the above sources that is indicative of a state of the user's well-being and could be useful in providing one or more recommendations to the user to affect or improve the user's well-being.

In any of the embodiments provided herein, the device input comprises data from one or more user devices. In any of the embodiments provided herein, the one or more user devices comprises physiological, neurological, psychological, metabolic, or biological data of the user. In any of the embodiments provided herein, the one or more user devices are configured to receive data associated with one or more of the following emotions of the user: alertness, excitement, elation, happiness, contentment, relaxation, calmness, sleepiness, fatigue, boredom, depression, sadness, frustration, stress, nervousness, tenseness, or a combination thereof.

In any of the embodiments provided herein, the one or more user devices comprises: one or more personal computing devices (e.g., a mobile device, tablet, or other personal computing device), one or more applications associated with the one or more personal computing devices (e.g., social media applications (e.g., Instagram, Facebook, Twitter, TikTok, Google Maps, Waze, Calendar, Microphone, Online Purchasing apps), one or more wearable devices (e.g., comprising one or more sensors for measuring a physiological state of the user (e.g., which may be connected to the one or more personal computing devices)), one or more implantable devices, one or more user connected devices, or a combination thereof. For example, the user device can include the user's cell phone, and the system can receive data from applications on the user's cell phone. For example, the system can receive data from applications such as Google Maps or Waze, to identify how long a user has been in the car in a given day. An extended period of time in the car (e.g., such as long commutes to and from work) can indicate a heightened sense of anxiety or stress in a user. As an additional example, the system can receive data from one or more social media applications. The system can receive data about how much interaction a user receives from their friends list or feed, how much interaction the user has with their friends list or feed, and how much time the user spends on social media. As an additional example, the system can receive data from the user's calendar which can show busy days at work, upcoming vacations, upcoming exams in school, and the like which are all event which can affect the user's well-being.

The one or more wearable devices can include smartwatches, wristbands, finger rings, glasses, gloves, headgear (such as hats, helmets, virtual reality headsets, augmented reality headsets, head-mounted devices (HMD), headbands), pendants, armbands, leg bands, shoes, vests, motion sensing devices, etc., or any other device capable of capturing and providing physiological, neurological, psychological, metabolic, or biological data of the user. The wearable device may be configured to be worn on a part of a user's body (e.g., a smartwatch or wristband may be worn on the user's wrist). The wearable device may be in communication with other devices (e.g., such as the user devices) and network 170.

The one or more implantable devices can include pacemakers, devices for measuring a user's blood glucose levels, devices for measuring blood alcohol levels, plasma drug (e.g., a 5-HT receptor agonist, medication, or over the counter drug) concentration levels, hormone levels, and/or metabolomics, and the like.

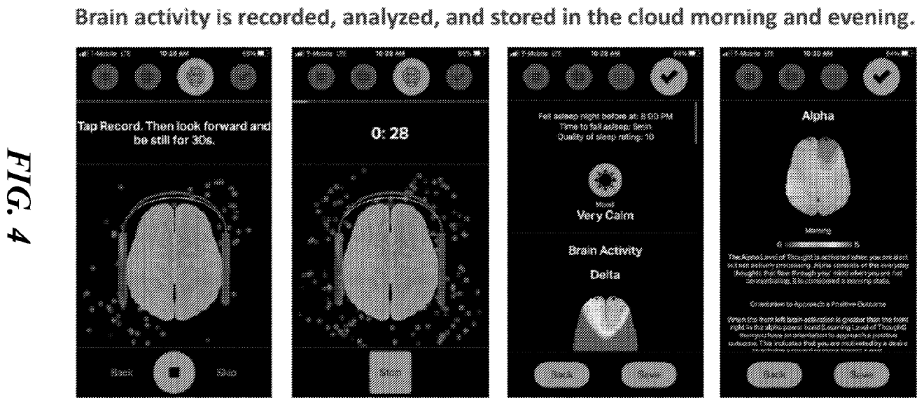

In any of the embodiments provided herein, receiving the data comprises performing an evaluation (e.g., a physiological (e.g., a brain) evaluation) on the user using the one or more wearable devices, or the one or more implanted devices. In any of the embodiments provided herein, the evaluation is performed at least once (e.g., twice, three times, four times, five times, six times, seven times, eight times, nine times, or ten times) daily to the user. In any of the embodiments provided herein, the evaluation is performed at least once (e.g., twice, three times, four times, five times, six times, seven times, eight times, nine times, or ten times) a week to the user. In any of the embodiments provided herein, the evaluation is performed at least once (e.g., twice, three times, four times, five times, six times, seven times, eight times, nine times, or ten times) a month to the user. FIG. 4 shows an example method instructing a user to use a wearable device for obtaining measurements of the user's brain activity. As shown, the brain activity can be associated and used to generate a visual representation to the user of the user's well-being (e.g., brain activity). The system can use the brain activity to identify a calm state of the user.

In any of the embodiments provided herein, the one or more wearable devices measures: a brain activity (e.g. using a brain evaluation (e.g. electroencephalogram (EEG), magnetoencephalogram (MEG), functional near-infrared spectroscopy (fNRIS), Positron Emission Tomography (PET) transcranial functional ultrasound)), a heart test (e.g. evaluated using an EEG for recording heart rate or electrocardiogram (EKG) for recording heart rhythm), a visual test, an auditory test, functional data (e.g. evaluated by brain imaging (e.g. fNRIS, PET) or monitoring of brain activity levels (e.g. EEG)), body temperature, food intake, metabolic rate, perspiration, hydration, salivation, pupil dilation, breathing rate, pulse rate, skin color, or skin temperature, and the like, of the user.

In any of the embodiments provided herein, the one or more wearable devices measures physiological, neurological, psychological, metabolic, or biological activity or a combination thereof of the user. In some embodiments the activity of a user is measured or identified by a brain activity (e.g. using a brain evaluation (e.g. EEG, MEG, fNRIS, PET transcranial functional ultrasound)), a heart test (e.g. evaluated using an EEG for recording heart rate or EKG for recording heart rhythm), a visual test, an auditory test, a biological sample (e.g. for evaluating changes in serum, plasma, whole blood, urine, sweat or the like), patient reporting (e.g. through questionnaires or surveys to evaluate mood, affect, coping, sleep quality, stress, anxiety, memory, and/or other emotions), functional data (e.g. evaluated by brain imaging (e.g. fNRIS, PET) or monitoring of brain activity levels (e.g. EEG)), body temperature, food intake, metabolic rate, perspiration, hydration, salivation, pupil dilation, breathing rate, pulse rate, skin color, or skin temperature, and the like. In any of the embodiments provided herein, the one or more wearable devices comprises an electroencephalogram (EEG) device (e.g., multi-electrode EEG device). In any of the embodiments provided herein, the EEG device comprises two or more electrodes that make contact with the forehead of the user. In any of the embodiments provided herein, the EEG device makes contact with the user's forehead for at least thirty seconds (e.g., at least 1, 2, 3, 4, 5, 6, 7, 8, 9, 10, 15, 20, 25, 30, 35, 40, 45, 50, 55, or 60 minutes). In any of the embodiments provided herein, the one or more wearable devices comprises one or more smart devices (e.g., smartwatches) worn by the user configured to measure physiological, neurological, psychological, metabolic, or biological data of the user.

In some embodiments, the activity of a user may be measured or identified by evaluating one or more of EEG, EKG, MEG, fNIRS, PET, transcranial functional ultrasound, visual stimuli (e.g. colour, shape, pattern, emotional face, video, flash, milli second or longer), auditory stimuli (e.g. matched paired, acoustic frequency, Hz, milli second or longer), ultrasound waves by a sensor e.g., a piezoelectric resonant material (e.g., a PZT, a CMUT, a PMUT), clinical effect (visual, auditory, body, time and space, cognition, drowsiness, confusion, impairment), standard/deviant waveforms, evoked power/potential, voltage (alpha bands, beta bands, gamma bands, delta bands, or theta bands), asynchronization, time-locked, magnetic, hemodynamic (flux, flow, velocity, oxygenation), MMN, ASSR, surveys or questionnaires (written or verbal) including 5D-ASC, Beck Depression, Coping, DASS42, Gad-7 Anxiety, PANAS-GEN, SF-36 QOL, STAI 6, brain imaging tests, heart rate, cardiovascular activity, photodiode, skin conduction and impedance, biological samples such as serum, plasma, whole blood, urine, sweat or the like, visual perception alteration, an auditory perception alteration, bodily perception alteration, a temporal perception alteration, or a spatial perception alteration, sleep quality, patient reported outcomes (e.g. electronic) or subjective mood, affect, coping, sleep quality, stress, anxiety, memory, and other emotional or functional data with brain imaging or brain activity, providing data (e.g., patterns, clusters, classifications, amplitudes, frequencies, or magnitude) patient evaluation (e.g., physicians, counselors, psychologist, or spiritual leader), data comprising visual representations of brain responses or brain activity data and/or survey or patient reported outcomes to help convey the effectiveness or lack thereof of a psychedelic therapy, training modules, machine learning algorithms and training sets, artificial intelligence (e.g., deep convolution neural networks) algorithms and/or machine learning processes to classify, cluster, or recognize patterns or relationships amongst sensory-evoked (e.g., auditory or visual stimuli) brain activity or resting state brain activity acquired by brain imaging methods and patient reported outcomes (e.g., changes in mood, anxiety, motivation, or memory), auditory brainstem responses (ABR), paired pulse inhibition (PPI; ie P50 auditory suppression; sensory gating), auditory mismatch negativity (MNN), and auditory steady state responses (ASSR), if EEG measures of auditory sensory evoked activity, cognitive control of attention and emotion, sub-thalamic and cortical levels, amplitudes of MMN potentials will be correlated to depression, anxiety, and mood survey data, Emotional Flanker Task, Erikson Flanker Task, Continuous Performance Test (CPT), Connor's CPT, oddball tasks, State Trait Anxiety Inventory is a 6-item, self-report survey, sleep diaries, the symmetry and power of alpha, beta, delta, and gamma brainwave activity across prefrontal cortex brain regions, Medical Quality of Life Outcomes Study 36-Item Short Form Health Survey (SF-36) and Physical Component Summary (PCS) and Mental Component Summary (MCS) scores, physical functioning, role physical, role emotional, bodily pain, vitality, social functioning, mental health, and general health on Likert, salivary cytokines, salivary brain-derived neurotrophic factors, psychophysical testing, assessing congruent, incongruent, and neutral conditions, surveys assessing mood, depression, anxiety, and stress, Mood Circumplex, DSM-V, cognitive behavioral therapy, synergy score, serenity score, quality of life, Sleep diary and Mood Capture, questions about attention, social interactions, fatigue and mood, approach avoid metric, biometric of coping, Heart Rate Variability (HRV) spot check, or camera.

In any of the embodiments provided herein, the sample input comprises data from one or more biological samples. In any of the embodiments provided herein, the one or more biological samples comprises user serum, plasma, tissue, whole blood, urine, fecal samples, sweat, or saliva. In any of the embodiments provided herein, data from one or more user biological samples comprises user blood glucose levels, blood alcohol levels, plasma drug (e.g., a 5-HT receptor agonist, medication, or over the counter drug) concentration levels, hormone levels, metabolomics, genetic information, or combinations thereof. For example, levels of one or more components (e.g., blood glucose level, alcohol level) in the blood can provide valuable insight as to the state of the user's well-being. In any of the embodiments provided herein, the one or more biological samples are obtained from a laboratory (e.g., clinical, hospital, bio bank, or outpatient laboratory), the user (e.g., at home sampling (e.g., of saliva, urine, fecal matter)), or a combination thereof. In any of the embodiments provided herein, the sample input comprises data related to one or more biomarkers of the user. In any of the embodiments provided herein, the data related to the one or more biomarkers of the user is identified from the one or more biological samples. In any of the embodiments provided herein, the one or more biomarkers are indicative of responsiveness to a treatment regimen. In any of the embodiments provided herein, the one or more biomarkers comprises inflammatory biomarkers (e.g., interleukins or cytokines), neurotransmitter metabolites, or a combination thereof. For example, the neurotransmitter metabolites can include metabolites of serotonin, dopamine, or the like. In any of the embodiments provided herein, the one or more biomarkers can include diagnostic, monitoring, pharmacodynamic/response, predictive, prognostic, safety, and/or susceptibility/risk biomarkers.

The system can process the data received from the one or more data sources to generate or extract one or more user specific parameters from the received data (220). In any of the embodiments provided herein, processing the data is performed using one or more data processing algorithms. For example, the data received from the one or more data sources and/or the database 130 can be processed for identifying information relevant to a user's well-being.

In any of the embodiments provided herein, the one or more data processing algorithms comprises one or more feature extraction algorithms, one or more machine learning algorithms, one or more artificial intelligence algorithms, one or more Bayesian algorithms (e.g., Bayesian assimilation), one or more statistical analysis algorithms, or a combination thereof. For example, feature extraction can reduce the number of features in a dataset described herein by creating new features from the existing ones (and then discarding the original features). The new reduced set of features can summarize the information contained in the original set of features. In this way, a summarized version of the original features can be created from a combination of the original set. The system can also implement feature selection. Feature selection can rank the importance of the existing features in the datasets described herein and discard less important ones. For example, feature extraction or feature selection can identify/rank the most relevant data associated with the user's well-being and provide that data for further processing for generating an output and one or more recommendations to the user for affecting or improving the user's well-being. This, and the other data processing algorithms described herein, can greatly lessen the computer resources (e.g., memory and storage) requirements for processing the databases described herein, which can be large and difficult to parse. The machine learning algorithms can implement classifiers (e.g., algorithms) and models for categorizing the databases described herein for identifying relevant information associated with a user's well-being. As an example, a machine learning algorithm can be used to categorize/identify data associated with a user's physical, mental, emotional, neurological, cognitive, and social disorders, increasing the efficiency of later models for generating outputs and one or more recommendations to the user for affecting or improving the user's well-being.

In any of the embodiments provided herein, processing the data is performed in a real-time, near real-time, or dynamic nature. In any of the embodiments provided herein, processing the data comprises batch processing. In any of the embodiments provided herein, the one or more data processing algorithms receive data from: (i) the one or more data sources, (ii) a database, or a combination thereof. In any of the embodiments provided herein, the database comprises stored reference population data, stored historical user specific data, or a combination thereof. In any of the embodiments provided herein, the one or more data processing algorithms comprise a natural language processing model configured to extract qualitative data from the one or more data sources, the historical user database, a reference population, or a combination thereof. The natural language processing model can be used to identify data including key terms or phrases associated with a user's physical, mental, emotional, neurological, cognitive, and social state, which state can be indicative of a user's overall well-being or health. In any of the embodiments provided herein, processing the received data further comprises processing the data from the historical user database, the reference population, or a combination thereof. In any of the embodiments provided herein, processing the data further comprises generating or extracting one or more labels from the reference population data. For example, the data processing algorithms can categorize data into physical, mental, emotional, neurological, cognitive, and social disorders subgroups of data and generate a label with the associated subgroup for improved efficiency for later modeling, generating outputs, and generating one or more recommendations for affecting or improving a user's well-being. For example, the data processing algorithms can categorize data into subgroups associated with one or more of the following emotions: alertness, excitement, elation, happiness, contentment, relaxation, calmness, sleepiness, fatigue, boredom, depression, sadness, frustration, stress, nervousness, tenseness, or a combination thereof, and generate a label with the associated subgroup for improved efficiency for later modeling, generating outputs, and generating one or more recommendations for affecting or improving a user's well-being. In any of the embodiments provided herein, processing the data further comprises identifying arbitrary data, data outliers (e.g., missing data or data falling outside a trend), or a combination thereof. For example, arbitrary data may be data that serves no use to the system. Data outliers can be data that falls outside of a given trend in the data (e.g., an observation that lies outside of an normal distance from other values in a data set). The outliers may comprise missing data. In any of the embodiments provided herein, the processing further comprises filling in missing data using one or more data interpolation methods. In any of the embodiments provided herein, updating the one or more models comprises updating the one or more labels based at least in part on the newly received data. In any of the embodiments provided herein, the method may further comprise removing arbitrary data from the dataset.

In any of the embodiments provided herein, the one or more user specific parameters comprises data associated with: drug use (e.g., a 5-HT receptor agonist, medication, over the counter drug, a dietary supplement, a plant-derivative substance, a natural product, a performance enhancer or a combination thereof) data, association with third parties (e.g., a doctor or therapist) data, emotional data, physical data, social interaction data, data related to one or more stressors, physiological data, neurological data, psychological data, metabolic data, or biological data. In any of the embodiments provided herein, the one or more user specific parameters comprises data associated with a: physical, mental, emotional, neurological, cognitive, and social state of a user. In any of the embodiments provided herein, the one or more labels identifies data in reference population associated with: drug use (e.g., a 5-HT receptor agonist, medication, over the counter drug, or a combination thereof) data, association with third parties (e.g., a doctor or therapist) data, emotional data, physical data, social interaction data, data related to one or more stressors, physiological data, neurological data, psychological data, metabolic data, or biological data. In any of the embodiments provided herein, the one or more labels identifies data in reference population associated with a: physical, mental, emotional, neurological, cognitive, and social state of a user. The physical, mental, emotional, neurological, cognitive, and social data may be as disclosed elsewhere herein. The physical, mental, emotional, neurological, cognitive, and social data may be derived from the one or more data sources, the reference population, or the historical user database. In any of the embodiments provided herein, the one or more user specific parameters comprises data associated with one or more of the following emotions: alertness, excitement, elation, happiness, contentment, relaxation, calmness, sleepiness, fatigue, boredom, depression, sadness, frustration, stress, nervousness, tenseness, or a combination thereof. In any of the embodiments provided herein, the one or more one or more labels identifies data in reference population associated with one or more of the following emotions: alertness, excitement, elation, happiness, contentment, relaxation, calmness, sleepiness, fatigue, boredom, depression, sadness, frustration, stress, nervousness, tenseness, or a combination thereof.

The system can initialize one or more models of well-being incorporating data from at least a reference population or a historical user database, and generating an output indicative of a state of the user's well-being based at least in part on the user specific parameters and the data from at least the reference population or the historical user database using the one or more models (230).

In any of the embodiments provided herein, the one or more models comprises one or more pre-programmed models. In any of the embodiments provided herein, the one or more models comprises one or more artificial intelligence models. In any of the embodiments provided herein, the one or more artificial intelligence models comprises one or more neuromorphic computing models. In any of the embodiments provided herein, the one or more neuromorphic computing models comprises a neural network (e.g., a spiking neural network). The one or more neuromorphic models can translate the functioning of the human brain into a computer system for performing any of the processes described herein. The one or more neuromorphic models can be parallel and handle many tasks (e.g., data processing tasks, modeling tasks, generating an output, generating one or more recommendations, or a combination thereof) at a given time. The one or more neuromorphic models can perform these tasks at a high computation speed and with low computational consumption (e.g., memory and storage consumption). The one or more neuromorphic models can adapt to different types of data received from the one or more data sources, the reference populations, and/or the historical user databases. The one or more neuromorphic models can have high energy efficiency, fault tolerance and powerful problem-solving capabilities. The one or more neuromorphic models can also solve novel problems and adapt to new environments very quickly. The one or more neuromorphic models can accomplish the tasks described herein by building artificial neural systems that implement “neurons” (e.g., nodes that process information) and “synapses” (e.g., connections between those nodes) to transfer electrical signals using analog circuitry. This can enable the one or more neuromorphic models to modulate the amount of electricity flowing between those nodes to mimic the varying degrees of strength that naturally occurring brain signals have. The system of neurons and synapses that transmit these electric pulses can be a spiking neural network (SNN), which can measure these discrete analog signal changes. The one or more neuromorphic models can also implement a chip architecture that collocates memory and processing together on each individual neuron instead of having separate designated areas for each. By collocating memory, a neuromorphic model/chip can process information efficiently.

In any of the embodiments provided herein, the one or more models comprises one or more machine learning models. In any of the embodiments provided herein, the one or more machine learning models comprises one or more artificial intelligence models. In any of the embodiments provided herein, the one or more machine learning models comprises a neural network (e.g., a spiking neural network, a deep neural network, a dynamic neural network, or a convolutional neural network), a regression-based learning algorithm, a linear or non-linear algorithm, a feed-forward neural network, a generative adversarial network (GAN), deep residual networks, a genetic algorithm, or any combination thereof.

In any of the embodiments provided herein, the one or more machine learning models comprises trained machine learning models. For example, the one or more machine learning models can be trained on a labeled set of data from the one or more data sources, the reference populations, and/or the historical user databases. The labeled set of data may identify the data in the data set associated with a user's physical, mental, emotional, neurological, cognitive, and social state. In any of the embodiments provided herein, the one or more machine learning models comprises supervised machine learning models, unsupervised machine learning models, or a combination thereof. In any of the embodiments provided herein, the one or more machine learning models compares the data received from the one or more data sources to the historical user database, the reference population data, or a combination thereof. In any of the embodiments provided herein, the one or more machine learning models generates an association between the user and the reference population data based at least in part on the one or more user specific parameters and the one or more labels. In any of the embodiments provided herein, the one or more machine learning models compares the data received from the one or more data sources to the reference population data using at least the association generated between the user and the reference population. In any of the embodiments provided herein, the output is generated based at least in part on the comparison of the data received from the one or more data sources to the reference population data. In any of the embodiments provided herein, the output is generated based at least in part on the comparison of the data received from the one or more data sources to historical user data stored on the database. In any of the embodiments provided herein, the output is generated in real-time, near real-time, or in a dynamic nature.

Examples of the machine learning models may comprise a regression-based learning algorithm, linear or non-linear algorithms, feed-forward neural network, generative adversarial network (GAN), or deep residual networks. The machine learning models may be, for example, unsupervised learning classifier, supervised learning classifier, or a combination thereof. The unsupervised learning classifier may be, for example, clustering, hierarchical clustering, k-means, mixture models, DBSCAN, OPTICS algorithm, anomaly detection, local outlier factor, neural networks, autoencoders, deep belief nets, hebbian learning, generative adversarial networks, self-organizing map, expectation-maximization algorithm (EM), method of moments, blind signal separation techniques, principal component analysis, independent component analysis, non-negative matrix factorization, singular value decomposition, or a combination thereof. The supervised learning classifier may be, for example, support vector machines, linear regression, logistic regression, linear discriminant analysis, decision trees, k-nearest neighbor algorithm, neural networks, similarity learning, or a combination thereof. In some embodiments, the machine learning models may comprise a deep neural network (DNN). The deep neural network may comprise a convolutional neural network (CNN). The CNN may be, for example, U-Net, ImageNet, LeNet-5, Alex Net, ZFNet, GoogleNet, VGGNet, ResNet18 or ResNet, etc. Other neural networks may be, for example, deep feed forward neural network, recurrent neural network, LSTM (Long Short Term Memory), GRU (Gated Recurrent Unit), Auto Encoder, variational autoencoder, adversarial autoencoder, denoising auto encoder, sparse auto encoder, boltzmann machine, RBM (Restricted BM), deep belief network, generative adversarial network (GAN), deep residual network, capsule network, or attention/transformer networks, etc. In some embodiments, the machine learning models may use a loss function. The loss function may be, for example, regression losses, mean absolute error, mean bias error, hinge loss, adam optimizer and/or cross entropy.

The one or models described above may be supervised machine learning algorithms. A supervised machine learning algorithm can be trained using labeled training inputs, i.e., training inputs with known outputs. For example, the machine learning model may be trained on known data from a reference population or a historical user database. The training inputs can be provided to an untrained or partially trained version of the machine learning algorithm to generate a predicted output. The predicted output can be compared to the known output, and if there is a difference, the parameters of the machine learning algorithm can be updated. A semi-supervised machine learning algorithm can be trained using a large number of unlabeled training inputs and a small number of labeled training inputs.

The one or more models described herein may be neural networks. Neural networks may employ multiple layers of operations to predict one or more outputs, e.g., the identity of a pictured product. Neural networks can include one or more hidden layers situated between an input layer and an output layer. The output of each layer can be used as input to another layer, e.g., the next hidden layer or the output layer. Each layer of a neural network can specify one or more transformation operations to be performed on input to the layer. Such transformation operations may be referred to as neurons. The output of a particular neuron can be a weighted sum of the inputs to the neuron, adjusted with a bias and multiplied by an activation function, e.g., a rectified linear unit (ReLU) or a sigmoid function.

Training a neural network can involve providing inputs to the untrained neural network to generate predicted outputs, comparing the predicted outputs to expected outputs, and updating the algorithm's weights and biases to account for the difference between the predicted outputs and the expected outputs. Specifically, a cost function can be used to calculate a difference between the predicted outputs and the expected outputs. By computing the derivative of the cost function with respect to the weights and biases of the network, the weights and biases can be iteratively adjusted over multiple cycles to minimize the cost function. Training may be complete when the predicted outputs satisfy a convergence condition, e.g., a small magnitude of calculated cost as determined by the cost function.

One type of neural network is a convolutional neural network (“CNN”). CNNs are neural networks in which neurons in some layers, called convolutional layers, receive pixels from only small portions of the input data set. These small portions may be referred to as the neurons' receptive fields. Each neuron in such a convolutional layer may have the same weights. In this way, the convolutional layer can detect features in any portion of the input data set. CNNSs may also have pooling layers that combine the outputs of neuron clusters in convolutional layers and fully-connected layers that are similar to traditional layers in a feed-forward neural network. CNNs may be particularly good at detecting and classifying object (e.g., products) in images.

In any of the embodiments provided herein, the one or more models comprises, one or more pre-programmed models, one or more artificial intelligence models, one or more machine learning models, or a combination thereof.

In any of the embodiments provided herein, the output is a score. In any of the embodiments provided herein, the score is a qualitative score, a quantitative score, or a combination thereof. For example, the score can be a detailed text outlining the need for one or more treatment regimen modification, one or more behavioral modification, or a combination thereof. As an additional example, the output can be a number indicating to the system to generate certain for one or more treatment regimen modification, one or more behavioral modification, or a combination thereof. In any of the embodiments provided herein, the score is positive, negative, or neutral. In any of the embodiments provided herein, the positive score is extremely positive, very positive, moderately positive, or slightly positive. In any of the embodiments provided herein, the negative score is extremely negative, very negative, moderately negative, or slightly negative. In any of the embodiments provided herein, the word positive can be interchangeable with happy, elated, alert, excited, content, relaxed, calm, or sleepy. In any of the embodiments provided herein, the word negative can be interchangeable with tense, nervous, stressed, frustrated, sad, depressed, bored, fatigued. In any of the embodiments provided herein, the score is a number on a scale from 0-100, wherein 0 indicates extremely negative, 50 indicates neutral, and 100 indicates extremely positive. In any of the embodiments provided herein, the score is a number on a scale from 0-10, wherein 0 indicates extremely negative, 5 indicates neutral, and 10 indicates extremely positive.

The system can update the one or more models based at least in part on newly received data from: (i) the one or more data sources, (ii) the historical user database, or (iii) the reference population (240). In any of the embodiments provided herein, wherein updating the one or more models comprises updating the one or more user specific parameters based at least in part on the newly received data. For example, as the data from the one or more data sources is being received, the user specific parameters can be updated in the one or more models based on the data as it is received to ensure a real-time, near real-time, or dynamic model of the user's well-being.

The system can generate one or more recommendations based at least in part on the output (250). In any of the embodiments provided herein, the method may further comprise generating one or more recommendations based at least in part on the output. In any of the embodiments provided herein, the one or more recommendations comprises providing a customized treatment regimen using one or more drugs to the user. For example, if a user is experiencing heightened states of depression, anxiety, and/or stress, etc., then the recommendation can include a treatment regimen of an anti-depressant or drug for reducing anxiety or stress. In any of the embodiments provided herein, the one or more drugs comprises a 5-HT receptor agonist, a prescribed medication, an over the counter drug, a dietary supplement, a plant-derivative substance, a natural product or a combination thereof. The 5-HT receptor agonist may comprise psilocybin. The 5-HT receptor agonist may comprise psilocin. The 5-HT receptor agonist may comprise LSD. The 5-HT receptor agonist may comprise any of the 5-HT receptor agonists disclosed herein. The prescribed medication can include one or more medications for affecting or improving a user's well-being (e.g., an anti-depressant).

In any of the embodiments provided herein, the customized treatment regimen comprises a dose modification. In any of the embodiments provided herein, the dose modification comprises a recommendation to increase or decrease a dose amount, a dose frequency, or a combination thereof, of at least one of the one or more drugs. For example, the output can indicate that a user is currently in a positive well-being state. The output can indicate that the user's well-being has increased significantly over a short period of time. In this case, the one or more recommendations can be to maintain the current dose, or decrease the dose.

In any of the embodiments provided herein, the one or more recommendations comprises one or more behavior modifications. In any of the embodiments provided herein, the one or more behavior modifications comprises a modification to: substance intake, physical exercise, diet, sleep schedule, cognitive behaviors, self-defeating behaviors, social interactions, compulsive behaviors, involuntary behaviors, addictions, or a combination thereof. For example, the one or more recommendations can be to increase the amount of physical exercise to affect or improve the user's well-being. The substance can be any addictive substance. The substance can be nicotine, alcohol, caffeine, soda, water, psychedelic, a dietary supplement, or a combination thereof. The self-defeating behaviors can include procrastination, physical/mental neglect, self-criticism, self-pity, perfectionism, comparing oneself to others, social withdrawal, refusing help, over-spending, relationship sabotage, over or under eating, self-inflicted harm, or drug and/or alcohol abuse. The compulsive behaviors can include addictive behaviors. The compulsive behaviors can include obsessive compulsive disorder (OCD). The compulsive behaviors can include shopping, eating, hoarding, gambling, skin picking (e.g., nail biting), sexual behavior, talking, or a combination thereof. The cognitive behavior can include recommending the user engage in cognitive behavior therapies. The involuntary behaviors can include nervous myoclonus or twitching (e.g., facial twitch), hiccups, etc.

In any of the embodiments provided herein, the one or more recommendations comprises providing a combination of a customized dosing regimen and one or more behavior modifications.

The system can provide a predicted efficacy associated with each of the one or more recommendations (260). In any of the embodiments provided herein, the method may further comprise providing a predicted efficacy associated with each of the one or more recommendations. The predicted efficacy can take into account the user's specific and personalized situation based on the output generation by the one or more models. For example, the efficacy can take into account the user's likelihood of being receptive to a specific drug. The efficacy can take into account a history of a user's behavior (e.g., the user seldomly or never exercises). The predicted efficacy can be a percentage. The percentage may be the percentage chance the one or more recommendations have at affecting or improving the user's well-being.

The system can generate a ranking for each of the one or more recommendations (270) In any of the embodiments provided herein, the method may further comprise generating a ranking for each of the one or more recommendations. In any of the embodiments provided herein, the predicted efficacy, ranking, or a combination thereof, is provided based on a probability density function. For example, the probability density function can identify a percentage number likelihood that the one or more recommendations is able to affect or improve a user's well-being at any given time. In any of the embodiments provided herein, the one or more recommendations are recommended if the one or more recommendations reach a threshold level of predicted efficacy. For example, the one or more recommendations will be thrown out if the predicted efficacy falls below 50%, 40%, 30%, 20%, or 10%. For example, the one or more recommendations will be displayed to the user and/or third parties if the predicted efficacy is above 50%, 60%, 70%, 80%, or 90%. In any of the embodiments provided herein, the ranking is based at least in part on the predicted efficacy. The ranking may also be based on the user's specific situation. For example, the system can take into account a user's likelihood of being receptive to a particular drug. The ranking can take into account a history of a user's behavior (e.g., the user seldomly or never exercises).

The system can display the one or more recommendations to the user on a graphical user interface (e.g., of a user device) (280). In any of the embodiments provided herein, the method may further comprise displaying the one or more recommendations to the user on a graphical user interface (e.g., of a user device). In any of the embodiments provided herein, the graphical user interface comprises a verbal recommendation. In any of the embodiments provided herein, the graphical user interface comprises a visual representation of the one or more recommendations.

In any of the embodiments provided herein, the visual representation comprises a representation of a human body comprising one or more highlighted body parts associated with the user's well-being. For example, the heart of a representation of a human body can be highlighted if the user's heart is in a positive or negative state. The color of the highlight can indicate the state of the body part. For example, red can indicate that action is needed to improve the current state of the highlighted body part. For example, green can indicate that no improvement is needed with the particular highlighted body part. For example, the heart can be highlighted red if physical exercise is needed to improve heart health. As an example, this recommendation can be generated based on data received from the one or more data sources indicating a high heart rate.

The system can provide the one or more recommendations to one or more third parties (e.g., a doctor, a therapist, fitness coach, wellness coach, lifestyle coach, spiritual guide, religious guide, or a combination thereof) associated with the user (280). For example, the system can provide the one or more recommendations to a user's doctor, who can then use the one or more recommendations for altering the doctor's course of treatment with the user.

The system can receive third party input to the one or more recommendations, and update the one or more recommendations based at least in part on the third party input (290). In any of the embodiments provided herein, the method may further comprise providing the one or more recommendations to one or more third parties (e.g., a doctor, a therapist, fitness coach, wellness coach, lifestyle coach, spiritual guide, religious guide, or a combination thereof) associated with the user. In any of the embodiments provided herein, the method may further comprise (i) receiving third party input to the one or more recommendations, and (ii) updating the one or more recommendations based at least in part on the third party input.

The system can simulate the user's well-being based at least in part on the user specific parameters and the data from the one or more data sources, the historical user database, or from the reference population (292). In any of the embodiments provided herein, the method may further comprises simulating the user's well-being based at least in part on the user specific parameters and the data from the one or more data sources, the historical user database, or from the reference population. In any of the embodiments provided herein, wherein the generating the output is further based at least in part on the simulation of the user's well-being. In any of the embodiments provided herein, the method may further comprise monitoring the user's well-being over a period of time.

The system can monitor the user's well-being over a period of time (294). In any of the embodiments provided herein, the monitoring comprising continuous monitoring or discrete monitoring. In any of the embodiments provided herein, the period of time comprises at least 1 day (e.g., 2, 3, 4, 5, 6, or 7 days). In any of the embodiments provided herein, the period of time comprises at least 1 week (e.g., 2, 3, or 4 weeks). In any of the embodiments provided herein, the period of time comprises at least 1 month (e.g., 2, 3, 4, 5, 6, 7, 8, 9, 10, 11, or 12 months). In any of the embodiments provided herein, the period of time comprises at least 1 year (e.g., 2, 3, 4, 5, 6, 7, 8, 9, 10, 11, or 12 years). For example, monitoring the user's well-being over the period of time can be useful for identifying one or more trends associated with the user's well-being. The one or more trends can be trends in user behavior. The one or more trends can be trends in how the user responds to certain recommended courses of action for affecting or improving the user's well-being. In any of the embodiments provided herein, the one or more recommendations is further based at least in part on a progress, digression, or combination thereof, of the user's well-being over the monitored period of time.

Provided in some embodiments herein is a method for monitoring the effectiveness of a 5-HT receptor agonist provided herein (e.g., psilocybin), comprising measuring (e.g., on a network (e.g., WiFi, cloud, BLE, 3/4/5G)) one or more brain response using a node or a wearable brain imaging device (e.g., an EEG). In some embodiments, the patient listens to paired or sustained auditory stimuli (e.g., at home, at work, or at school). In some embodiments, the auditory stimuli being delivered from an application on a (e.g., mobile) device (e.g., cellular phone, tablet, or the like).

Provided in some embodiments herein is a method for using Bayesian statistics and methods to estimate and predict confidence in the effectiveness of a treatment or dose of a psychedelic compound provided herein based on historical brain imaging data in a database for particular patient types.

Provided in some embodiments herein is a method of imaging brain activity (e.g., voltage) in an individual using a wearable device (e.g., EEG (e.g., multi-electrode EEG device)), the wearable device having two or more electrodes that make contact with the forehead of the individual for at least thirty seconds and up to twenty minutes.

Provided in some embodiments herein is a method for optically imaging brain activity in an individual (e.g., from a wearable device) to determine, track, or optimize the effectiveness of a psychedelic compound, wherein a brain signal is identified by transmission of visible light (400-680 nanometers) or infrared light (greater than or equal to 680 nanometers) across the skin into the brain to collect reflected photons by a sensor (e.g., a photodiode or CMOS).

Provided in some embodiments herein is a method of acoustically imaging brain activity in an individual (e.g., from a wearable device) to determine, track, or optimize the effectiveness of a psychedelic compound, wherein a brain signal is identified by transmission of ultrasound (e.g., having an acoustic frequency of greater than or equal to one megahertz) (e.g., across the skin and skull) into the brain to collect reflected ultrasound waves by a sensor (e.g., a piezoelectric resonant material (e.g., a PZT, a CMUT, a PMUT).

In some embodiments, the method further comprises using artificial intelligence (e.g., deep convolution neural networks) algorithms and/or machine learning processes to classify, cluster, or recognize patterns or relationships amongst sensory-evoked (e.g., auditory or visual stimuli) brain activity or resting state brain activity acquired by brain imaging methods and patient reported outcomes (e.g., changes in mood, anxiety, motivation, or memory) for optimizing the dosing schedule or treatment paradigm or a patient undergoing treatment with psychedelic substances (e.g. psilocybin).

Provided in some embodiments herein is a computer-implemented method for identifying a therapeutically effective dose of a 5-HT receptor agonist administered to an individual, the method comprising:
a) administering the 5-HT receptor agonist to the individual;
b) emitting one or more (e.g., auditory or visual) to the individual;
c) receiving, from a brain imaging device (e.g., an EEG), a brain response; and
d) identifying the therapeutically effective dose of the 5-HT receptor agonist based at least in part on the one or more (e.g., auditory or visual) stimulus and the brain response.

In some embodiments the method further comprises receiving, from the individual, an emotional data, and wherein the therapeutically effective dose of the 5-HT receptor agonist is further determined based on the emotional data. In some embodiments, the emotional data comprises a mood rating, a sleep rating, a stress rating, an anxiety rating, a memory rating, or any combination thereof.

In some embodiments, two or more of (a), (b), and (c) are performed simultaneously.

In some embodiments, two or more of (a), (b), and (c) are performed sequentially.

In some embodiments, the method further comprises transmitting the therapeutically effective dose of the 5-HT receptor agonist to the individual, a caregiver, or both.

In some embodiments, the therapeutically effective dose of the 5-HT receptor agonist is transmitted by a mobile device (e.g., cellular phone, tablet). In some embodiments, the therapeutically effective dose of the 5-HT receptor agonist is transmitted over a wireless network (e.g., WiFi, cloud, BLE, 3/4/5G). In some embodiments, the therapeutically effective dose of the 5-HT receptor agonist is determined by a machine learning algorithm.

Provided in some embodiments herein is a computer-implemented system for identifying a therapeutically effective dose of a 5-HT receptor agonist administered to an individual, the system comprising: a digital processing device comprising: at least one processor, an operating system configured to perform executable instructions, a memory, and a computer program including instructions executable by the digital processing device to create an application configured to perform at least the following:
a) receiving an indicator of the 5-HT receptor agonist administered to the 5-HT receptor agonist provided to the individual;
b) directing a device (e.g., an auditory or visual device) to emit one or more (auditory or visual) stimulus to the individual;
c) receiving, from a brain imaging device (e.g., EEG), a brain response; and
d) identifying the therapeutically effective dose of the 5-HT receptor agonist based at least in part on the one or more (auditory or visual) stimulus and the brain response.

In some embodiments, the application is further configured to perform receiving, from the individual, an emotional data, and wherein the therapeutically effective dose of the 5-HT receptor agonist is further determined based on the emotional data. In some embodiments, the emotional data comprises a mood rating, a sleep rating, a stress rating, an anxiety rating, a memory rating, or any combination thereof. In some embodiments, the application is configured to simultaneously perform two or more of steps (a), (b), and (c). In some embodiments, the application is configured to sequentially perform two or more of steps (a), (b), and (c).

In some embodiments, the application is further configured to transmit the therapeutically effective dose of the 5-HT receptor agonist to the individual, a caregiver, or both. In some embodiments, the application directs the transmission of the therapeutically effective dose of the 5-HT receptor agonist by a mobile device (e.g., cellular phone, tablet). In some embodiments, the application directs the transmission of the therapeutically effective dose of the 5-HT receptor agonist over a wireless network (e.g., WiFi, cloud, BLE, 3/4/5G). In some embodiments, the therapeutically effective dose of the 5-HT receptor agonist is determined by a machine learning algorithm.

Provided in some embodiments herein is non-transitory computer-readable storage media encoded with a computer program including instructions executable by a processor to create an application for identifying a therapeutically effective dose of a 5-HT receptor agonist administered to an individual, the application configured to perform at least the following:
a) receiving an indicator of the 5-HT receptor agonist administered to the individual;
b) directing a device (e.g., an auditory device or a visual device) to emit one or more (e.g., auditory or visual) stimulus to the individual;
c) receiving, from a brain imaging device (e.g., EEG), a brain response; and
d) identifying the therapeutically effective dose of the 5-HT receptor agonist based at least in part on the auditory stimulus and the brain response.

In some embodiments, the application is further configured to perform receiving, from the individual, an emotional data, and wherein the therapeutically effective dose of the 5-HT receptor agonist is further determined based on the emotional data. In some embodiments, the emotional data comprises a mood rating, a sleep rating, a stress rating, an anxiety rating, a memory rating, or any combination thereof.

In some embodiments, the application is configured to simultaneously perform two or more of steps (a), (b), and (c). In some embodiments, the application is configured to sequentially perform two or more of steps (a), (b), and (c).

In some embodiments, the application is further configured to transmit the therapeutically effective dose of the 5-HT receptor agonist to the individual, a caregiver, or both. In some embodiments, the application directs the transmission of the therapeutically effective dose of the 5-HT receptor agonist by a mobile device (e.g., cellular phone, tablet). In some embodiments, the application directs the transmission of the therapeutically effective dose of the 5-HT receptor agonist over a wireless network (e.g., WiFi, cloud, BLE, 3/4/5G). In some embodiments, the therapeutically effective dose of the 5-HT receptor agonist is determined by a machine learning algorithm.

Provided in some embodiments herein is a computer-implemented system for identifying a therapeutically effective dose of a 5-HT receptor agonist administered to an individual, the system comprising: a digital processing device comprising: at least one processor, an operating system configured to perform executable instructions, a memory, and a computer program including instructions executable by the digital processing device to create an application configured to perform at least the following:
a) applying a first machine learning algorithm to a plurality of 5-HT receptor agonist doses (e.g., dose responses), a plurality of (e.g., auditory or visual) stimuli, and brain responses to identifying the therapeutically effective dose of the 5-HT receptor agonist;
b) receiving verified emotional data regarding an emotional effectiveness of the 5-HT receptor agonist; and
c) feeding back the verified data to improve the first machine learning algorithm's calculation over time.

In some embodiments, the first machine learning algorithm is trained by a neural network comprising:
a) a first training module for creating a first training set comprising a set of 5-HT receptor agonist doses (e.g., dose responses), each dose associated with one (e.g., auditory or visual) stimulus, and one brain response;
b) a first training module training the neural network using the first training set;
c) a second training module creating a second training set for second stage training comprising the first training set and the psychedelic treatments incorrectly detected as having a positive effectiveness on the individual after the first stage of training; and
d) training the neural network using the second training set.

Provided in some embodiments herein is a non-transitory computer-readable storage media encoded with a computer program including instructions executable by a processor to create an application for identifying a therapeutically effective dose of a 5-HT receptor agonist administered to an individual, the application configured to perform at least the following:
a) applying a first machine learning algorithm to a plurality of 5-HT receptor agonist doses (e.g., dose responses), a plurality of (e.g., auditory or visual) stimuli, and brain responses to identify the therapeutically effective dose of the 5-HT receptor agonist;
b) receiving verified emotional data regarding an emotional effectiveness of the 5-HT receptor agonist; and
c) feeding back the verified data to improve the first machine learning algorithm's calculation over time.

In some embodiments, the first machine learning algorithm is trained by a neural network comprising:
a) a first training module creating a first training set comprising a set of 5-HT receptor agonist doses (e.g., dose responses), each dose associated with one (e.g., auditory or visual) stimulus, and one brain response;
b) a first training module training the neural network using the first training set;
c) a second training module creating a second training set for second stage training comprising the first training set and the 5-HT receptor agonist doses (e.g., dose responses) incorrectly detected as having a positive effectiveness on the individual after the first stage of training; and
d) training the neural network using the second training set.

In some embodiments, the application can be used to monitor brain activity of subjects. The application can pair to a subject's device (e.g., EEG device). A subject can log onto the application using their subject ID. The application can be designed to acquire data regarding stress and anxiety using a modified version of the Short State Trait Anxiety Inventory (STAI-6) once per day. An example of a STAI-6. The application can collect data on mood using a modified version of the Russel Mood Circumplex twice daily. The application can record information about sleep time and quality, fatigue, and drug use in the morning and evening using a modified version of the NIH Sleep Diary.

The application can work with a custom engineered, at home EEG device (e.g., a CGX, LLC device) that records activity from the prefrontal cortex using a wearable, low-risk device. The application can pair automatically to the device using Bluetooth. The rechargeable, battery-powered, wearable EEG device can use disposable, biocompatible, snap biopotential sensor electrodes. Users can record resting state at any time they wish in one-minute epochs. The option to record resting state brain activity in the morning and evening can be built into the questionnaires. Subjects may be asked to record 1-minute epochs of resting state EEG activity in the morning or evening at least 4 times per week. Subjects can record resting state EEG activity as often as desired.

The application can connect patients and users to medical and recreational marijuana.

The application can record information about sleep time and quality, fatigue, and drug use in the morning and evening using a modified version of the NIH Sleep Diary.

The application can also record information about coping. For example, the application can utilize an Approach-Avoid metric to measure coping.

In some embodiments, auditory stimuli comprises auditory tasks. Auditory tasks may comprise P50 paired click auditory suppression, Mismatch Negativity (MMN), and/or Auditory Steady State Response (ASSR). The P50 task can involve the subject listening to 120 paired auditory clicks (1 msec each) occurring 500 msec apart. Each pairing can occur 10 sec apart. The estimated time to complete the passive P50 task is 20 min. The ASSR task can require subjects to listen to a series of tones using a 500 or 1000 Hz carrier frequency modulated at 20 or 40 Hz for 1 second each. Each 1 sec tone can be separated by a three second inter-stimulus interval. A total of 100 tones at 20 and 40 Hz each can be delivered. The ASSR task can be expected to last 15 minutes. The MMN task can require subjects to listen to 500 tones (1 msec) spaced 1 second apart. There can be 400 tones at a standard frequency (750 Hz) and 100 randomly spaced odd-ball tones (1500 Hz). The MMN task can be expected to take 10 minutes. Subjects can also undergo a final resting state period of EEG collection where they lay down with their eyes closed for 10 minutes. EEG recordings will also include other biosensors for recording heart rate activity during testing.

In some embodiments, visual stimuli may include one or more visual tasks comprising an Emotional Flanker Task and/or a Continuous Performance Test (CPT). Following EEG assessments following auditory tasks as described above, subjects can undergo a series of computerized cognitive attention and emotional tasks while recording EEG. Subjects can first complete an Emotional Flanker Test that uses neutral faces or an angry face as a background distractor on classic flanker stimuli assessing congruent, incongruent, and neutral conditions. A stimulus from each condition can be presented 50 times and a total of 250 stimuli can be presented over 10 minutes. Subjects can then take a Conner's Continuous Performance Test that lasts 14 minutes to assess attention and cognitive control.

### Computer Systems

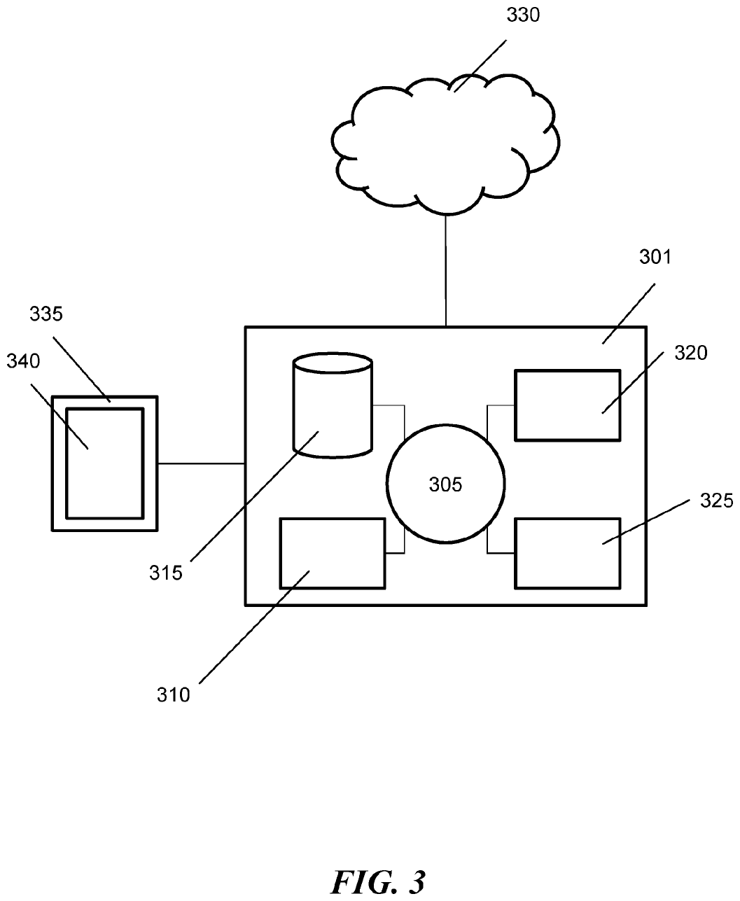

The present disclosure provides computer systems that are programmed to implement methods of the disclosure. FIG. 3 shows a computer system 301 that is programmed or otherwise configured to affect or improve a user's well-being. The computer system 301 can be an electronic device of a user or a computer system that is remotely located with respect to the electronic device. The electronic device can be a mobile electronic device.

The computer system 301 includes a central processing unit (CPU, also “processor” and “computer processor” herein) 305, which can be a single core or multi core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communication bus (solid lines), such as a motherboard. The storage unit 315 can be a data storage unit (or data repository) for storing data. The computer system 301 can be operatively coupled to a computer network (“network”) 330 with the aid of the communication interface 320. The network 330 can be the Internet, an internet and/or extranet, or an intranet and/or extranet that is in communication with the Internet. The network 330 in some cases is a telecommunication and/or data network. The network 330 can include one or more computer servers, which can enable distributed computing, such as cloud computing. The network 330, in some cases with the aid of the computer system 301, can implement a peer-to-peer network, which may enable devices coupled to the computer system 301 to behave as a client or a server.

The CPU 305 can execute a sequence of machine-readable instructions, which can be embodied in a program or software. The instructions may be stored in a memory location, such as the memory 310. The instructions can be directed to the CPU 305, which can subsequently program or otherwise configure the CPU 305 to implement methods of the present disclosure. Examples of operations performed by the CPU 305 can include fetch, decode, execute, and writeback.

The CPU 305 can be part of a circuit, such as an integrated circuit. One or more other components of the system 301 can be included in the circuit. In some cases, the circuit is an application specific integrated circuit (ASIC).

The storage unit 315 can store files, such as drivers, libraries and saved programs. The storage unit 315 can store user data, e.g., user preferences and user programs. The computer system 301 in some cases can include one or more additional data storage units that are external to the computer system 301, such as located on a remote server that is in communication with the computer system 301 through an intranet or the Internet.

The computer system 301 can communicate with one or more remote computer systems through the network 330. For instance, the computer system 301 can communicate with a remote computer system of a user (e.g., a database, an enterprise or extraprise system, an Internet-of-Things (IoT) device, a sensor, or the like). Examples of remote computer systems include personal computers (e.g., portable PC), slate or tablet PC's (e.g., Apple® iPad, Samsung Galaxy Tab), telephones, Smart phones (e.g., Apple® iphone, Android-enabled device, Blackberry®), or personal digital assistants. The user can access the computer system 301 via the network 330.

Methods as described herein can be implemented by way of machine (e.g., computer processor) executable code stored on an electronic storage location of the computer system 301, such as, for example, on the memory 310 or electronic storage unit 315. The machine executable or machine readable code can be provided in the form of software. During use, the code can be executed by the processor 305. In some cases, the code can be retrieved from the storage unit 315 and stored on the memory 310 for ready access by the processor 305. In some situations, the electronic storage unit 315 can be precluded, and machine-executable instructions are stored on memory 310.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 301 can include or be in communication with an electronic display 335 that comprises a user interface (UI) 340 for providing, for example, displaying one or more recommendations to a user, a third party associated with a user, or both. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 305. The algorithm can, for example, process data for identifying user specific parameters. The algorithm can, for example, process reference population data, historical user data for identifying data relevant to well-being. The algorithm can, for example, Initialize one or more models of well-being incorporating data from at least a reference population or a historical user database, and generating an output indicative of a state of the user's well-being based at least in part on the user specific parameters and the data from at least the reference population or the historical user database using the one or more models.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-131. (canceled)

132. A method for affecting a user's well-being, the method comprising:
(a) receiving data from one or more data sources;
(b) processing the data received from the one or more data sources to generate or extract one or more user specific parameters from the received data; and
(c) initializing one or more models of well-being incorporating data from at least a reference population or a historical user database, and generating an output indicative of a state of the user's well-being based at least in part on the user specific parameters and the data from at least the reference population or the historical user database using the one or more models.

133. The method of claim 132, wherein affecting the user's well-being comprises improving the user's well-being or improving the user's health outcomes.

134. The method of claim 132, wherein the reference population data, the historical user data, or a combination thereof, are gathered over a time period of at least 1 day, at least 1 week, at least 1 month, or at least 1 year.

135. The method of claim 132, wherein:
(a) the reference population data, the historical user database, or a combination thereof, are received from a third party library;
(b) the reference population data is gathered by: (i) receiving data from one or more data sources associated with a reference population, and (ii) processing the data received from the one or more data sources; and/or
(c) the one or more user specific parameters comprises data associated with: drug use data, third-party data, emotional data, physical data, social interaction data, data related to one or more stressors, physiological data, neurological data, psychological data, metabolic data, or biological data.

136. The method of claim 132, further comprising:
(a) updating the one or more models based at least in part on newly received data from: (i) the one or more data sources, (ii) the historical user database, or (iii) the reference population, optionally wherein updating the one or more models comprises updating the one or more user specific parameters based at least in part on the newly received data; and/or
(b) generating one or more recommendations based at least in part on the output.

137. The method of claim 136, wherein:
the one or more recommendations comprises providing a customized treatment regimen using one or more drugs to be taken by the user, wherein the customized treatment comprises, or does not comprise, a dose modification;
the one or more recommendations comprises one or more behavior modifications, such as, a modification to: substance intake, physical exercise, diet, sleep schedule, cognitive behaviors, self-defeating behaviors, social interactions, compulsive behaviors, involuntary behaviors, addictions, or a combination thereof; and/or
the one or more recommendations are generated in real time, near real time or in a dynamic nature.

138. The method of claim 136, further comprising:
(a) providing a predicted efficacy associated with each of the one or more recommendations, optionally wherein the predicted efficacy is provided based on a probability density function and/or wherein the one or more recommendations are recommended if the one or more recommendations reach a threshold level of predicted efficacy;
(b) generating a ranking for each of the one or more recommendations, optionally wherein the ranking is based at least in part on the predicted efficacy;
(c) displaying the one or more recommendations to the user on a graphical user interface, optionally wherein the graphical user interface comprises a verbal recommendation and/or a visual representation of the one or more recommendations;
(d) providing the one or more recommendations to one or more third parties associated with the user; and/or
(e) receiving third party input to the one or more recommendations and updating the one or more recommendations based at least in part on the third-party input.

139. The method of claim 132, wherein each of the one or more data sources is independently selected from the group consisting of user input, third party input, device input, and sample input.

140. The method of claim 139, wherein:
(a) the user input comprises data received from one or more user devices;
(b) receiving the data comprises administering one or more surveys or questionnaires to the user, or the user input comprises user responses to the one or more surveys or questionnaires;
(c) the user input comprises quantitative input, qualitative input, or a combination thereof; and/or
(d) the user input comprises the user's unprompted input related to the user's well-being, which optionally comprises a self-examination of the user's well-being.

141. The method of claim 140, wherein:
the one or more surveys or questionnaires are administered at least once daily, at least once a week, or at least once a month to the user;
the one or more surveys or questionnaires comprises surveys acquiring emotional data of the user, wherein the emotional data relates to depression, anxiety, stress, coping, mood, sleep, attention, quality of life, phobias, demoralization, rumination, social interaction, or a combination thereof, of the user;
the one or more surveys or questionnaires comprises surveys acquiring physical data of the user, wherein the physical data comprises data relating to the user's physiological state, physical exercise, substance consumption, or a combination thereof;
the one or more surveys or questionnaires comprises surveys acquiring data of one or more stressors of the user, wherein the one or more stressors comprises data relating to the user's daily interactions and/or to long term stress associated with family, social life, financial status, health, accidents, response to news, or a combination thereof; and/or
the one or more surveys or questionnaires comprises a Beck Depression Inventory (BDI), a Generalized Anxiety Disorder (GAD-7), a Depression Anxiety Stress Scale (DASS), a Brief-COPE, a Positive and Negative Affect Schedule (PANAS), a State Trait Anxiety Inventory (STAI), a modified version of a Russel Mood Circumplex, a modified version of a NIH Sleep Diary, a 36 Item Short Form Health Survey (SF-36), a 5D Altered state of Consciousness Scale (5d-ASC), a daily sleep diary questionnaire, a Hamilton Rating Scale for Depression, a Hamilton Anxiety Rating Scale, mood surveys, self-reporting surveys, sleep diaries or a combination thereof.

142. The method of claim 139, wherein:
(a) the third party input comprises:
data from the user's doctor(s), family, friends, co-workers, therapists, counselors, teachers, professors, spiritual guides, religious guides, religious guides, fitness coach, wellness coach, lifestyle coach, healthcare workers, or a combination thereof;
doctor input on the user's well-being; and/or
input from the user's family, friends, co-workers, therapists, and/or counselors; and/or
(b) the device input comprises data from one or more user devices, wherein:
the one or more user devices comprises physiological, neurological, psychological, metabolic, or biological data of the user; and/or
the one or more user devices comprises: one or more personal computing devices, one or more applications associated with the one or more personal computing devices, one or more wearable devices, one or more implantable devices, one or more user connected devices, or a combination thereof.

143. The method of claim 142, wherein receiving the data comprises performing an evaluation on the user using the one or more wearable devices, or the one or more implanted devices, wherein:
(a) the evaluation is performed at least once daily, at least once a week, or at least once a month to the user;
(b) the one or more wearable devices measures: a brain activity, a heart test, a visual test, an auditory test, functional data or monitoring of brain activity levels, body temperature, food intake, metabolic rate, perspiration, hydration, salivation, pupil dilation, breathing rate, pulse rate, skin color, or skin temperature, or the like, of the user;
(c) the one or more wearable devices comprises an electroencephalogram (EEG) device; and/or
(d) the one or more wearable devices comprises one or more smart devices worn by the user configured to measure physiological, neurological, psychological, metabolic, or biological data of the user.

144. The method of claim 139, wherein the sample input comprises:
(a) data from one or more biological samples, wherein:
the one or more biological samples comprises user serum, plasma, tissue, whole blood, urine, fecal samples, sweat, or saliva;
the data from the one or more user biological samples comprises user blood glucose levels, blood alcohol levels, plasma drug concentration levels, hormone levels, metabolomics, genetic information, or combinations thereof; and/or
the one or more biological samples are obtained from a laboratory, the user, or a combination thereof; and/or
(b) data related to one or more biomarkers of the user, wherein:
the data related to the one or more biomarkers of the user is identified from the one or more biological samples;
the one or more biomarkers are indicative of responsiveness to a treatment regimen;
the one or more biomarkers comprises inflammatory biomarkers, neurotransmitter metabolites, or a combination thereof; and/or
the one or more biomarkers comprise diagnostic, monitoring, pharmacodynamic/response, predictive, prognostic, safety, and/or susceptibility/risk biomarkers.

145. The method of claim 132, wherein processing the data:
(a) is performed using one or more data processing algorithms, optionally wherein:
the one or more data processing algorithms comprises one or more feature extraction algorithms, one or more machine learning algorithms, one or more artificial intelligence algorithms, one or more Bayesian algorithms, one or more statistical analysis algorithms, or a combination thereof;
the one or more data processing algorithms receive data from: (i) the one or more data sources, (ii) a database, or a combination thereof; and/or
the one or more data processing algorithms comprise a natural language processing model configured to extract qualitative data from the one or more data sources, the historical user database, a reference population, or a combination thereof
(b) is performed in a real-time, near real-time, or dynamic nature;
(c) comprises batch processing;
(d) comprises processing the data from the historical user database, the reference population, or a combination thereof;
(e) comprises generating or extracting one or more labels from the reference population data, optionally wherein the one or more labels identifies data in reference population associated with: drug use data, association with third parties data, emotional data, physical data, social interaction data, data related to one or more stressors, physiological data, neurological data, psychological data, metabolic data, or biological data;
(f) comprises identifying arbitrary data, data outliers, or a combination thereof; and/or
(g) comprises filling in missing data using one or more data interpolation methods.

146. The method of claim 132, wherein the one or more models comprises:
(a) one or more pre-programmed models;
(b) one or more artificial intelligence models, optionally wherein the one or more artificial intelligence models comprises one or more neuromorphic computing models; and/or
(c) one or more machine learning models.

147. The method of claim 146, wherein the one or more machine learning models:
comprises one or more artificial intelligence models;
comprises a neural network, a regression-based learning algorithm, a linear or non-linear algorithm, a feed-forward neural network, a generative adversarial network (GAN), deep residual networks, a genetic algorithm, or any combination thereof;
comprises trained machine learning models;
comprises supervised machine learning models, unsupervised machine learning models, or a combination thereof;
compares the data received from the one or more data sources to the historical user database, the reference population data, or a combination thereof;
generates an association between the user and the reference population data based at least in part on the one or more user specific parameters and the one or more labels; or
compares the data received from the one or more data sources to the reference population data using at least the association generated between the user and the reference population.

148. The method of claim 132, wherein the output:
(a) is generated based at least in part on the comparison of the data received from the one or more data sources to the reference population data;
(b) is generated based at least in part on the comparison of the data received from the one or more data sources to historical user data stored on the database;
(c) is generated in real-time, near real-time, or in a dynamic nature;
(d) is a score, which is (i) a qualitative score, a quantitative score, or a combination thereof, (ii) positive, negative or neutral and/or (iii) a number on a scale from 0-100, wherein 0 indicates extremely negative, 50 indicates neutral, and 100 indicates extremely positive.

149. The method of claim 132, further comprising:
(a) simulating the user's well-being based at least in part on the user specific parameters and the data from the one or more data sources, the historical user database, or from the reference population, optionally wherein the generating the output is further based at least in part on the simulation of the user's well-being; and/or
(b) monitoring the user's well-being over a period of time, wherein the monitoring comprises continuous monitoring or discrete monitoring and/or one or more recommendations are generated based at least in part on a progress, digression, or combination thereof, of the user's well-being over the monitored period of time.

150. One or more non-transitory computer storage media storing instructions that are operable, when executed by one or more computers, to cause the one or more computers to perform operations comprising:
(a) receiving data from one or more data sources;
(b) processing the data received from the one or more data sources to generate or extract one or more user specific parameters from the received data; and
(c) initializing one or more models of well-being incorporating data from at least a reference population or a historical user database, and generating an output indicative of a state of the user's well-being based at least in part on the user specific parameters and the data from at least the reference population or the historical user database using the one or more models.

151. A system comprising one or more computers and one or more storage devices storing instructions that are operable, when executed by the one or more computers, to cause the one or more computers to perform operations comprising:
(a) receiving data from one or more data sources;
(b) processing the data received from the one or more data sources to generate or extract one or more user specific parameters from the received data; and
(c) initializing one or more models of well-being incorporating data from at least a reference population or a historical user database, and generating an output indicative of a state of the user's well-being based at least in part on the user specific parameters and the data from at least the reference population or the historical user database using the one or more models.
